SEQUENCE LISTING (34 sequences)

SEQ ID NO: 1 - DNA, 1617 bp, Artificial Sequence
  cDNA of human HERV-W
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 2 - DNA, 1617 bp, Artificial Sequence
  cDNA of human HERV-FRD
atgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaa

SEQ ID NO: 3 - DNA, 1854 bp, Artificial Sequence
  cDNA murine Syncytin A
atggttcgtccttgggttttctgtctcctcctgttcccttgttcctctgcctactcggacagctggatgcccctggtaaacctcactcaacacctcctccaggaggctaactcttccttctcttccaactgctgggtctgcttatccatccaaacccagcgctctctagccatgccagccccactaaggacttggacagagacacccatgaaacttcgaatcatgtactcagcccggaccctctccggcccataccctatcaccgaccttgagaggcgcctccagaatttccaaccattgactccccactcctcttttgtcaaccctgaccagcgggccattgctttccttcagatcaccagcgtgacaggcatacttcccatactttctcggatcacctcggtgagataccccgatgaccacgtctatgaatctgcccagcgccccatatggggctcactctccacccagacgatcctcacctcccaggcccctctctgcatatcccgcttcttcaagaattcaaaccatgccaccttcgtgggcaaactccctgcctctctttgcaatcacacctttcagctctccccctctgccaaccaccaatccatagatctgtcctccagctatgcattcgccccattaatggccatgccagggtctaaatggagaaaccccttacgcttttcaggacccccttccctgaactcagggatgcctcactactcctgcccgatagatgacatccactgccacacctaccccaccaccccctggaggtcctgtccttccttcccagctagcacctgctataatctcaccctattcgagccggacaattcgagccaccctattaccctgtctgtggacaccacatacttcaagattaaactccagggacacaaagacccctatccactctttcagtaccagcccctcatgggggcagccctctctggacaatattcaatctgggaatatgaacccactgttaagaaaaacggcggtatcactccaaatatcttctcccaccttgtctccttaacatactccttctgcctcaactcctctggtgttttcttcctctgtggaaactcaacttatgtctgtctcccggccaattggtccggcgtctgtacccttgtcttccaatacccggatattgaactccttcccaacaaccaaaccatatctgtcccgctttttgctacagttccctcctctgtccccgcttctcgccggaagcgagcccttcctctccttcctctcctcgcgggcctgggcattgcttctgccctggggttaggcatcgcgggtatcaccacctcaactgtgtatttccaacagctttccaaggctctctcggacagcctagatgaaatagccacctccatcatcagcctccaagaccaaatagactcgctggcgggtgtcgttctccaaaaccgcagagctctggacctcattgtggctgagagggggggcacctgcctcttcctccaggaagagtgctgcttctacataaaccagtccggggtagtccggcacgcggcaaggaaacttcgagaaagggcctcggaactcggcacaagctcgagttcttggatccagtggctggggctaggaccctggctgccctcttggttgacttccctcatggctcccattctctttatcctggtactgctggttttcaggccttgtcttcttaactgcctgactcattctgtatcgcggcgaatgagttctttcattcacaccaccaccgaaggacacgtggacaagatccttctgcttcgagagtcccagtacaagagacttccccaagagcccccggaggaggatgccgtctag

SEQ ID NO: 4 - DNA, 1857 bp, Artificial Sequence
  cDNA murine Syncytin B
atgacaggcttttgggtcctctgtttcgtccttttcccctcctccttatcctatccggaaagctggatgccccttgtaaacctcactcaccacatcctacgtgataccaactcttccctgttttccaactgttgggtctgcttgtctacccaaacccagcggtccttagcagtcccagcccctctgtccatttggacagatacacccatgaagcttcatcttacctactcagtcaggcccttctctggctccttttccattagcgacattgaaagacgcctccgtctcttccgcccactgactgcctcctattctttccacaatcctgacagaagggcgattgcttttcttcaactcgtcagctcaacaggcatatttcggatcatcacccggataacctctgtgatatatccccataaggaccgtttcttcgaatctgcccaacgccctctctggggaccactctttactgagaccgtgctcaggtcgcaggccccactctgcatatctcgctttttcaaggtctcagcatatgccacttttgtaggcaacctctctgcctctctctgcaactacaccatgcatatttcaccttctaccagtcatgaaaacctagatctttccaccacccatacgttcaaacaggcaatgaaaagaccggatgccaaatggaaaaacccgctccgtttttccgggcccccctccctcatcttctcgaagccggcttactatccctgcccaacagacatcaaacactgccatacctctccggccactccctggatgcactgtcctcaggctcccttcggcacctgctataacctcactttatttgaaccagacaactcaacccaccctgttaccatgtcagtgaaccctacccacttcaaggtcaaactccaggggcacagagacccctatccgctctcccattaccagcccctcacgggagctgccctgtctggacaatattcagtctgggagaacgagatcactgtccaagaaaactgggacatcacctccaacattttctcacatcttctcagcttctcgtacgccttctgcctcaactcttcaggcgttttcttcctctgcggaacatcgacttacatctgcctcccagccaattggtccggtgtctgtaccctggtcttccaatacccggatattgaacttctccccaataaccaaacggtgcctgttcccctttttgcttcagttctttcctcagactcagttcttcgcccaaagaggtcccctcacctctttcccttccttgcaggcctgggtatctcttctgcccttggtacggggatagctggcttggccacctcgactctctatttccaacagctttctaaggttctttccgaaaccttggaagaaatagctgcctctatcactaccctccagaaccaaatagactcgctcgcaggtgttgttctacaaaaccgccgagctctggacctcatcactgctgagaaagggggcacctgtctcttcctccaggaagagtgctgcttctacgtaaaccagtctggaatagtccgggacgcggcaaggaaactccaagaacgagcatctgaactcggccagcattctgactcttggggacagtggcctgaccttggacgttggttgccctggctgactccctttctgggacctcttctcttcctcttcttcctactgacatttgggtcttgtcttctgaactgcctaacccgttttgtgtcccagagacttggctcctttgttcaagacactgccaaaaggcatgtggacagcatcctccaaaatttccaatataaaaaactgccccaagactccccagatgaggacaccattcctacataa

SEQ ID NO: 5 - DNA, 7150 bp, Artificial Sequence
  pcDNA3.1 SYNCYTIN-1
tcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagc

SEQ ID NO: 6 - DNA, 7134 bp, Artificial Sequence
  pcDNA3.1 SYNCYTIN-2
cactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttat

SEQ ID NO: 7 - DNA, 20 bp, Artificial Sequence
  PSI forward
caggactcggcttgctgaag

SEQ ID NO: 8 - DNA, 20 bp, Artificial Sequence
  PSI reverse
tcccccgcttaatactgacg

SEQ ID NO: 9 - DNA, 19 bp, Artificial Sequence
  PSI probe (FAM)
cgcacggcaagaggcgagg

SEQ ID NO: 10 - DNA, 21 bp, Artificial Sequence
  TitinMex5 forward
aaaacgagcagtgacgtgagc

SEQ ID NO: 11 - DNA, 20 bp, Artificial Sequence
  TitinMex5 reverse
ttcagtcatgctgctagcgc

SEQ ID NO: 12 - DNA, 25 bp, Artificial Sequence
  Titin probe (VIC)
tgcacggaagcgtctcgtctcagtc

SEQ ID NO: 13 - DNA, 20 bp, Artificial Sequence
  WPRE forward
ggcactgacaattccgtggt

SEQ ID NO: 14 - DNA, 20 bp, Artificial Sequence
  WPRE reverse
agggacgtagcagaaggacg

SEQ ID NO: 15 - DNA, 23 bp, Artificial Sequence
  WPRE probe (FAM)
acgtcctttccatggctgctcgc

SEQ ID NO: 16 - DNA, 22 bp, Artificial Sequence
  Ex20ext primer
cagaattctgccaattgatgag

SEQ ID NO: 17 - DNA, 20 bp, Artificial Sequence
  Ex26ext primer
ttcttcagcttgtgtcatcc

SEQ ID NO: 18 - DNA, 24 bp, Artificial Sequence
  Ex20int primer
cccagtctaccaccctatcagagc

SEQ ID NO: 19 - DNA, 20 bp, Artificial Sequence
  Ex26int primer
cctggctttaaggcttcctt

SEQ ID NO: 20 - DNA, 19 bp, artificial sequence
  sunthetic oligonucleotide primer Psi-F
agcctcaataaagcttgcc

SEQ ID NO: 21 - DNA, 19 bp, artificial sequence
  synthetic oligonucleotide primer RRE-R
tctgatcctgtcgtaaggg

SEQ ID NO: 22 - DNA, 18 bp, artificial sequence
  synthetic oligonucleotide primer AAV-forward
ccaggcgaggagaaacca

SEQ ID NO: 23 - DNA, 25 bp, artificial sequence
  synthetic oligonucleotide primer AAV-reverse
cttgactccactcagttctcttgct

SEQ ID NO: 24 - DNA, 29 bp, artificial sequence
  synthetic oligonucleotide probe AAV
ctcgccgtaaaacatggaaggaacacttc

SEQ ID NO: 25 - DNA, 20 bp, artificial sequence
  synthetic oligonucleotide primer 920hasarco;F
tgctggcctatgtcatgtgc

SEQ ID NO: 26 - DNA, 20 bp, artificial sequence
  synthetic oligonucleotide primer 991hasarco.R
tctggatgtcggaggtagcc

SEQ ID NO: 27 - DNA, 25 bp, artificial sequence
  synthetic oligonucleotide probe 946hasarco.P
cgggagggaaggctgaagagagacc

SEQ ID NO: 28 - DNA, 20 bp, artificial sequence
  synthetic oligonucleotide primer 0181P0.F
ctccaagcagatgcagcaga

SEQ ID NO: 29 - DNA, 20 bp, artificial sequence
  synthetic oligonucleotide primer m267P0.R
accatgatgcgcaaggccat

SEQ ID NO: 30 - DNA, 22 bp, artificial sequence
  synthetic oligonucleotide probe m225P0.P
ccgtggtgctgatgggcaagaa

SEQ ID NO: 31 - DNA, 20 bp, artificial sequence
  synthetic oligonucleotide primer mLy6e forward
cgggctttgggaatgtcaac

SEQ ID NO: 32 - DNA, 20 bp, artificial sequence
  synthetic oligonucleotide primer mly6e reverse
gtgggatactggcacgaagt

SEQ ID NO: 33 - DNA, 20 bp, artificial sequence
  synthetic oligonucleotide primer PO reverse
ctccaagcagatgcagcaga

SEQ ID NO: 34 - DNA, 20 bp, artificial sequence
  synthetic oligonucleotide primer PO forward
accatgatgcgcaaggccat

(19) United States
(12) Patent Application Publication
(10) Pub. No.: US 2020/0261589 A1
(43) Pub. Date: Aug. 20, 2020

(54) Use of a Syncytin for Targeting Drug and Gene Delivery to Regenerate Muscle Tissue

(71) Applicants: Genethon, Evy (FR); Institut National de la Santé et de la Recherche Médicale, Paris (FR); Université d'Evry val d'Essonne, Evy (FR)

(72) Inventors: Anne Galy, Fontainebleau (FR); Maxime Ferrand, Mennecy (FR)

(73) Assignees: Genethon, Evry (FR); Institut National de la Santé et de la Recherche Médicale, Paris (FR); Université d'Evry val d'Essonne, Evry (FR)

(21) Appl. No.: 16/757,591

(22) PCT Filed: Oct. 19, 2018
(86) PCT No.: PCT/EP2018/078804

§ 371 Date: Apr. 20, 2020

(30) Foreign Application Priority Data
Oct. 20, 2017 (EP) 17306448.6

(51) Int. Cl. A61K 47/64; A61K 9/127; A61K 45/06; A61K 9/00; A61K 35/76; A61K 47/69; A61P 21/00

(52) U.S. Cl. CPC A61K 47/64; A61K 9/127; A61K 45/06; A61P 21/00; A61K 35/76; A61K 47/6901; A61K 9/0019

(57) ABSTRACT

The invention relates to a pharmaceutical composition for targeting drug delivery including gene delivery to regenerating muscle tissue, comprising at least a therapeutic drug or gene, associated to a syncytin protein, and its use in the prevention and/or treatment of muscle injuries or diseases, in particular in gene therapy of said diseases using lentiviral vector particles or lentivirus-like particles pseudotyped with syncytin protein.

### FIELD OF THE INVENTION

The present invention relates to pharmaceutical compositions for targeting regenerating muscle tissue and to their use in the prevention and/or treatment of muscle injuries or diseases. More particularly, the present invention relates to the use of syncytin for targeting drug delivery including gene delivery to regenerating muscle tissue via injection.

### BACKGROUND OF THE INVENTION

Gene therapy might provide a cure for many different types of myopathies of genetic origin, but this approach is proving to be a difficult endeavor. There are many difficulties with gene transfer to skeletal muscle. Various vectors tested in muscle have proven to be immunogenic and at present, only the non-inflammatory recombinant Adeno-Associated Vectors (rAAVs) remain in use in preclinical and clinical studies aiming at gene transfer in muscle. These rAAVs remain episomal in the target cells and as they do not integrate they cannot be transmitted in replicating cells. This mode of action is useful for gene transfer in differentiated post-mitotic tissues such as adult skeletal muscle fibers, but may not permit long-term gene expression in muscle progenitor cells with high proliferation potential or in muscle tissue undergoing highly-regenerative processes. A second limitation of rAAVs is its small cargo capacity, currently limited to 4.5 Kb, which prevents the use of this vector system for large genes such as dystrophin. In addition, while rAAV is not an inflammatory vector, it is nonetheless capable of inducing strong immune responses to its viral capsid as demonstrated in preclinical models and in clinical trials. Re-administration of rAAV of the same serotype is currently not possible unless immunosuppressive treatments are administered to patients and this is not always possible in the benefit/risk analysis of gene therapy. So, there is a need for additional, novel, more physiological gene therapy vectors, with a high cargo capacity and that could permit gene transfer into regenerating muscle or muscle progenitor cells.

Lentiviral vectors (LV) which are enveloped RNA particles measuring approximately 120 nm in size are efficient drug delivery tools and more particularly efficient gene delivery tools for stable long-term transduction. The LV binds to, and enters into target cells through its envelope proteins which confer its pseudotype. Once the LV has entered into the cells, it releases its capsid components and undergoes reverse transcription of the lentiviral RNA before integrating permanently the proviral DNA into the genome of target cells. Thus, LV enables stable gene transfer into replicating cells. Non-integrative lentiviral vectors have been generated by modifying the properties of the vector integration machinery and can be used for transient gene expression. Virus-like particles lacking a provirus have also been generated and can be used to deliver proteins or messenger RNA. LV can be used for example, for gene addition, RNA interference, exon skipping or gene editing. All of these approaches can be facilitated by tissue or cell targeting of the LV via its pseudotype.

The most commonly-used pseudotype for LV is the G glycoprotein of vesicular stomatitis virus (VSVg). The broad tropism of VSVg enables ubiquitous gene delivery to many different types of cells in vitro. LV-VSVg are mostly used ex vivo in the case of hematopoietic gene therapy or to generate CAR T cells. LV are also used in vivo in a few applications for which small amounts of vector are administered to the brain or the eye. Systemic administration of LV-VSVg is usually not done because these vectors are known to be immunogenic in vivo in mice. Indeed, in vivo VSVg binds complement and when used in vivo targets transgene delivery to the liver and lymphoid organs triggering anti-transgene immune responses (Ciré et al. Plos One 9, e101644, 2014). Thus, there is a need for new pseudotypes for LV able to provide stable in vivo gene delivery without loss of transgene-expressing cells. This could be useful for gene transfer into muscle, in particular regenerating muscle tissue and muscle progenitor cells. Indeed LV have a large cargo capacity and recently it has been shown that the dystrophin cDNA (11 kb) could be fitted into a LV cassette (Counsell et al. Sci. Report, 2017, 7:46880. doi: 10.1038), providing a possible strategy to treat all Duchenne Muscular Dystrophy patients.

Syncytin, are endogenous retroviral virus (ERV syncytins) envelope glycoproteins which have fusogenic properties (Dupressoir et al., Proceedings of the National Academy of Sciences of the United States of America, 2005, 102, 725-730; Lavialle et al., Phil. Trans. R. Soc. B., 2013, 368:20120507). Human endogenous retroviral envelope glycoprotein encoded by the ERVW-1 gene (ENSG00000242950; also known as syncytin-1 or HERV-W) has been described for its fusogenic properties in patent application EP2385058. Said application describes its use in cancer treatment, by the formation of syncytia. Murine syncytins encompasse murine syncytin-A (i.e.: Mus musculus syncytin-A, synA) and murine syncytin-B (i.e.: Mus musculus syncytin-B, synB).

It has been shown recently that murine syncytins are expressed in the skeletal muscle and in particular that syncytin B is important for muscular fiber regeneration in male mice but not in female mice, for as yet unexplained reasons (Redelsperger et al., PLOS Genetics, 2016, 12(9): e1006289. doi: 10.1371).

It was also reported that syncytin does not generate functional pseudotypes, probably due to improper incorporation into viral particles (Bacquin et al., J. Virol., 2017, 91(18):e00832-17. doi:10.1128).

### SUMMARY OF THE INVENTION

Surprisingly, and contrary to what would be expected from the prior art, the inventors have found that syncytin may be used to pseudotype LV and as such may be used for targeting stable gene delivery in regenerating muscle tissue without diffusing to other organ, thereby avoiding risk of liver toxicity.

Indeed the murine syncytin-A glycoprotein was used to pseudotype a HIV-1-derived lentiviral vectors encoding several transgene sequences: either the luciferase LucII to facilitate the detection of transgene expression by bioluminescence, or a small antisense sequence for dystrophin exon 23 skipping (U7mex23) or human alpha sarcoglycan gene to show a functional effect. The pseudotyped LVs were injected intramuscularly to mice with normal skeletal muscle (C57B16), mdx mice deficient in dystrophin, a model of Duchenne Muscular Dystrophy with highly regenerative skeletal muscle fibers, and alpha-sarcoglycan-deficient mice which are undergoing muscle regeneration. By comparing the effects of the Syncytin A-pseudotypes LV (LV-SynA) in these different models, it was shown that LV pseudotyped with Syncytin A preferentially transduce muscle that is undergoing regeneration. By contrast, injection of LV-SynA directly into muscle does not lead to a significant transduction of skeletal muscle tissue in normal mice. The transduction of regenerating muscle by LV-SynA cannot be predicted from in vitro data using murine myoblast cells (C2C12) commonly used as model of myoblast to myotube differentiation. Indeed, stable transgene expression was reproducibly obtained in regenerating muscle cells for long periods of time, at least 50 days, with no expression in the liver. In contrast, LV pseudotyped with other envelopes such as VSVg provide only temporary expression. In addition, LV-SynA vectors are less immunogenic than LV-VSVg as they induced less transgene specific immune responses following intramuscular or systemic administration. Furthermore, evidence of induction of dystrophin exon skipping was obtained in mdx mice with the syncytin-A LV vectors. In vivo correction of gene deficiency of sgca-deficient mice is feasible by gene transfer with LV-SynA Sgca vector and the expression of the therapeutic transgene can be enhanced by repeated injections of vector in the same muscle. LV pseudotyped with human syncytins such as Syncytin2 could be used to transduce human skeletal muscle to express a transgene stably.

These results provide the proof-of-concept that syncytin can be reliably used for targeted delivery of a therapeutic drug such as a therapeutic gene or a gene encoding a therapeutic drug to regenerating muscle tissue, in particular for gene therapy of myopathies such as with no limitation Duchenne Muscular Dystrophy and limb-girdle muscular dystrophies, using lentiviral vector particles pseudotyped with syncytin.

By comparison rAAV, although it was injected intramuscularly, disseminated much beyond muscle and was found at high levels in the liver. Furthermore, in vivo gene delivery with LV-Syncytin is expected to be more stable than with episomal rAAV due to the integrative nature of the LV vector and the lower immunogenicity of LV pseudotyped with syncytin. Moreover, LV have a larger cargo capacity than rAAV and can incorporate large transgenes such as dystrophin cDNA. In view of all these advantages, LV pseudotyped with syncytin represent a very promising alternative to rAAV for gene therapy of myopathies.

Thus the present invention relates to a pharmaceutical composition for targeting regenerating muscle tissue, comprising at least a drug associated to a syncytin protein, for use in the prevention and/or treatment of muscle injuries or diseases.

### DETAILED DESCRIPTION OF THE INVENTION

Syncytins (also named ERV syncytins) according to the invention refer to highly fusogenic envelope glycoproteins from eutherian mammals, which belong to the family of Endogenous Retroviruses (ERVs). These proteins are encoded by genes, which display a preferential expression in placenta and induce syncytium formation when introduced into cultured cells (Lavialle et al., Phil. Trans. R. Soc. B., 2013, 368:20120507).

Syncytins according to the invention can be selected from human syncytins (e.g.: HERV-W and HERV-FRD), murine syncytins (e.g.: syncytin-A and syncytin-B), syncytin-Ory1, syncytin-Car1, syncytin-Rum1 or their functional orthologs (Dupressoir et al., Proceedings of the National Academy of Sciences of the United States of America, 2005, 102, 725-730; Lavialle et al., Phil. Trans. R. Soc. B., 2013, 368:20120507), and functional fragments thereof comprising at least the receptor binding domain (corresponding to residues 117-144 of Syncytin-1).

By functional orthologs it is intended ortholog proteins encoded by ortholog genes and that exhibit fusogenic properties. Fusogenic properties may be assessed in fusion assays as described in Dupressoir et al. (PNAS 2005). Briefly, cells are transfected for example by using Lipofectamine (Invitrogen) and about 1-2 μg of DNA for 5×10⁵ cells or calcium phosphate precipitation (Invitrogen, 5-20 μg of DNA for 5×10⁵ cells). Plates are generally inspected for cell fusion 24-48 h after transfection. Syncytia can be visualized by using May—Grünwald and Giemsa staining (Sigma) and the fusion index calculated as [(N—S)/T]×100, where N is the number of nuclei in the syncytia, S is the number of syncytia, and T is the total number of nuclei counted.

Human syncytins encompasses HERV-W and HERV-FRD. Functional orthologs of these proteins can be found in Hominidae. HERV-W refers to a highly fusogenic membrane glycoprotein belonging to the family of Human Endogenous Retroviruses (HERVs). HERV-W is an envelope glycoprotein; it is also called Syncytin-1. It has the sequence indicated in Ensembl database, corresponding to Transcript ERVW-1-001, ENST00000493463. The corresponding cDNA has the sequence listed in SEQ ID NO:1. HERV-FRD also refers to a highly fusogenic membrane glycoprotein belonging to the family of Human Endogenous Retroviruses (HERVs). HERV-FRD is an envelope glycoprotein, also called Syncytin-2. It has the sequence indicated in Ensembl database, corresponding to Transcript ERVFRD-1, ENSG00000244476. The corresponding cDNA has the sequence listed in SEQ ID NO:2. Murine syncytins encompasses murine syncytin-A (i.e.: Mus musculus syncytin-A, synA) and murine syncytin-B (i.e.: Mus musculus syncytin-B, synB). Functional orthologs of these proteins can be found in the Muridae family. Murine syncytin-A is encoded by the syncytin-A gene. Syncytin-A has the sequence indicated in Ensembl database Syna ENSMUSG00000085957. The corresponding cDNA has the sequence listed in SEQ ID NO:3. Murine syncytin-B is encoded by the syncytin-B gene. Syncytin-B has the sequence indicated in Ensembl databaseSynb ENSMUSG00000047977. The corresponding cDNA has the sequence listed in SEQ ID NO: 4.

The syncytin-Ory1 is encoded by the syncytin-Ory1 gene. Functional orthologs of syncytin-Ory1 can be found in the Leporidae family (typically rabbit and hare).

The syncytin-Car1 is encoded by the syncytin-Car1 gene. Functional orthologs of syncytin-Car1 can be found in carnivores mammals from the Laurasiatheria superorder (Cornelis et al., Proceedings of the National Academy of Sciences of the United States of America, 2013, 110, E828-E837; Lavialle et al., Phil. Trans. R. Soc. B., 2013, 368:20120507).

The syncytin-Rum1 is encoded by the syncytin-Rum1 gene. Functional orthologs of syncytin Rum-1 can be found in ruminant mammals.

In the various embodiments of the present invention, the syncytin according to the invention can be typically selected from the group consisting of HERV-W (Syncytin-1), HERV-FRD (Syncytin-2), syncytin-A, syncytin-B, syncytin-Ory1, syncytin-Car1 and syncytin-Rum1 and their functional orthologs; preferably the syncytin is selected from the group consisting of HERV-W, HERV-FRD, murine syncytin-A, murine syncytin-B and their functional orthologs, more preferably the syncytin is selected from the group consisting of HERV-W, HERV-FRD murine syncytin-A and murine syncytin-B. In some preferred embodiments, the syncytin is syncytin-A, Syncytin-1 or Syncytin-2; preferably syncytin-A or Syncytin-2.

In the various embodiments of the present invention, the therapeutic drug is associated to a syncytin protein, directly or indirectly, via covalent or not covalent coupling or bonding using standard coupling methods that are known in the art.

In some embodiments, the drug is covalently coupled to the syncytin protein. For example, the drug can be conjugated to syncytin. Covalent coupling of the drug to syncytin may be achieved by incorporating a reactive group in syncytin protein, and then using the group to link the drug covalently. Alternatively a drug which is a protein can be fused to syncytin to form a fusion protein wherein the syncytin and drug amino acid sequences are linked directly or via a peptide spacer or linker.

In some other embodiments, the drug and syncytin protein are incorporated into a drug delivery vehicle, such as for example a polymer-based or particle-based delivery vehicle including with no limitations micelle, liposome, exosome, dendrimer, microparticle, nanoparticle, virus particle, virus-like particle and others.

As used herein, the term “viral vector” refers to a non-replicating, non-pathogenic virus engineered for the delivery of genetic material into cells. In viral vectors, viral genes essential for replication and virulence have been replaced with heterogeneous gene of interest.

As used herein, the term “recombinant virus” refers to a virus, in particular a viral vector, produced by recombinant DNA technology.

As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins.

As used herein, the term “Virus Like Particle” or “VLP” refers to self-assembling, non-replicating, non-pathogenic, genomeless particle, similar in size and conformation to intact infectious virus particle.

In some preferred embodiments, the drug and syncytin protein are incorporated into particles such as for example liposomes, exosomes, microparticles, nanoparticles, virus particles and virus-like particles. The particles are advantageously selected from the group consisting of liposomes, exosomes, virus particles and virus-like particles. Virus particles and virus-like particles include viral capsids and enveloped virus or virus-like particles. Enveloped virus or virus-like particles include pseudotyped virus or virus-like particles. The virus or virus-like particles are preferably from a retrovirus, more preferably a lentivirus. The virus particles are advantageously from a viral vector, preferably a lentiviral vector.

Retrovirus includes in particular gammaretrovirus, spumavirus, and lentivirus. Lentivirus includes in particular human immunodeficiency virus such as HIV type 1 (HIV1) and HIV type 2 (HIV2) and equine infectious anemia virus (EIAV).

Lentivirus-like particles are described for example in Muratori et al., Methods Mol. Biol., 2010, 614, 111-24; Burney et al., Curr. HIV Res., 2006, 4, 475-484; Kaczmarczyk et al., Proc. Natl. Aca. Sci; U.S.A., 2011, 108, 16998-17003; Aoki et al., Gene Therapy, 2011, 18, 936-941. Examples of lentivirus-like particles are VLPs generated by co-expressing in producer cells, a syncytin protein with a gag fusion protein (Gag fused with the gene of interest).

The drug and/or syncytin may be, either displayed on the surface of the particles, or enclosed (packaged) into the particles. The syncytin protein is advantageously displayed on the surface of the particles, such as coupled to the particles or incorporated into the envelope of (enveloped) virus particles or virus-like particles to form pseudotyped enveloped virus particles or virus-like particles. The drug is coupled to the particles or packaged into the particles. For example, the drug is coupled to viral capsids or packaged into viral capsids, wherein said viral capsids may further comprise an envelope, preferably pseudotyped with syncytin. In some preferred embodiments, the drug is packaged into particles pseudotyped with syncytin protein. The drug which is packaged into particles is advantageously a (heterologous) gene of interest which is packaged into viral vector particles, preferably retroviral vector particles, more preferably lentiviral vector particles.

In some more preferred embodiments, the particles are enveloped virus particles or virus-like particles, preferably enveloped virus particles or virus-like particles pseudotyped with syncytin protein, even more preferably lentivirus vector particles pseudotyped with syncytin protein or lentivirus-like particles pseudotyped with syncytin protein. The enveloped virus particles pseudotyped with syncytin protein, preferably lentivirus vector particles pseudotyped with syncytin protein are advantageously packaging a (heterologous) gene of interest. In some preferred embodiments, the lentivirus vector particles, preferably packaging a (heterologous) gene of interest, are pseudotyped with syncytin-A, Syncytin-1 or Syncytin-2; preferably syncytin-A or Syncytin-2.

In the various embodiments of the present invention, muscle injuries or muscle diseases (myopathies) include regeneration phases as part of the disease physiopathological process. The drug is any drug of interest for treating the muscle injuries or diseases by targeted delivery to the cells of the regenerating muscle tissue, in particular myocytes, myotubes, myoblasts, and/or satellite cells and more preferably myotubes, myoblasts, and/or satellite cells. Such drugs include any drug capable of stimulating muscle regeneration, in particular skeletal muscle regeneration such as with no limitations: growth factors and prostaglandine anti-inflammatory drugs; immunotherapeutic drugs including immunomodulatory, immunosuppressive, anti-histaminic, anti-allergic or immunostimulating drugs; anti-infectious drugs such as anti-bacterial, viral, fungal or parasitic drugs; anti-cancer drugs; therapeutic proteins including therapeutic antibodies or antibody fragments and genome-editing enzymes, therapeutic peptides, therapeutic RNAs and genes of interest for therapy of muscular diseases or injuries including therapeutic genes and genes encoding therapeutic proteins, therapeutic peptides, and/or therapeutic RNAs as listed above. The drug may be a natural, synthetic or recombinant molecule or agent, such as a nucleic acid, peptide nucleic acid (PNA), protein including antibody and antibody fragment, peptide, lipid including phospholipid, lipoprotein and phospholipoprotein, sugar, small molecule, other molecule or agent, or a mixture thereof. Immunosuppressive drugs include for example interleukin 10 (IL10), CTLA4-Ig and other immunosuppressive proteins or peptides. Therapeutic antibodies include for instance antibodies against myostatin. Therapeutic nucleic acids such as therapeutic RNAs include antisense RNAs capable of exon skipping such as modified small nuclear RNAs (snRNAs), guide RNAs or templates for gene editing, and interfering RNAs such as shRNAs and microRNAs.

By “gene of interest for therapy”, “gene of therapeutic interest”, “gene of interest” or “heterologous gene of interest”, it is meant a therapeutic gene or a gene encoding a therapeutic protein, peptide or RNA for treating muscle injuries or diseases including regeneration phases as part of the disease physiopathological process.

The therapeutic gene may be a functional version of a gene or a fragment thereof. The functional version or variant includes the wild-type version of said gene, a variant gene belonging to the same family, or a truncated version, which preserves the functionality of the encoded protein. A functional version of a gene is useful for replacement or additive gene therapy to replace a gene, which is deficient or non-functional in a patient. A fragment of a functional version or variant of a gene is useful as recombination template for use in combination with a genome editing enzyme.

Alternatively, the gene of interest may encode a therapeutic protein including a therapeutic antibody or antibody fragment, a genome-editing enzyme or a therapeutic RNA. The gene of interest is a functional gene able to produce the encoded protein, peptide or RNA in cells of the regenerating muscle tissue, in particular myocytes, myotubes, myoblasts, and/or satellite cells and more preferably myotubes, myoblasts, and/or satellite cells. The therapeutic protein may be any drug capable of stimulating muscle regeneration as defined above.

The therapeutic RNA is advantageously complementary to a target DNA or RNA sequence. For example, the therapeutic RNA is an interfering RNA such as a shRNA, a microRNA, a guide RNA (gRNA) for use in combination with a Cas enzyme or similar enzyme for genome editing or an antisense RNA capable of exon skipping such as a modified small nuclear RNA (snRNA). The interfering RNA or microRNA may be used to regulate the expression of a target gene involved in muscle disease. The guide RNA in complex with a Cas enzyme or similar enzyme for genome editing may be used to modify the sequence of a target gene, in particular to correct the sequence of a mutated/deficient gene or to modify the expression of a target gene involved in muscle disease. The antisense RNA capable of exon skipping is used in particular to correct a reading frame and restore expression of a deficient gene having a disrupted reading frame.

The genome-editing enzyme according to the invention is an enzyme or enzyme complex that induces a genetic modification at a target genomic locus. The genome-editing enzyme is advantageously an engineered nuclease which generates a double-strand break (DSB) in the target genomic locus, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease, is usually but not necessarily used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a muscle disease.

The gene of interest is advantageously packaged into an enveloped viral vector particle pseudotyped with syncytin protein, preferably a lentivirus vector particle pseudotyped with syncytin protein. The viral vector comprises the gene of interest in a form expressible in muscle cells. In particular, the gene of interest is operatively linked to a ubiquitous, tissue-specific or inducible promoter which is functional in muscle cells such as the Spleen Focus Forming Virus (SFFV) promoter or the synthetic muscle-specific promoter C5-12 (Wang et al., Gene Therapy, 2008, 15, 1489-1499).

In some preferred embodiments of the present invention, the drug of interest including a gene of interest for treating muscular injuries or diseases is specific for muscle diseases in that it targets a gene or gene product (protein/peptide) involved in muscle disease(s) that is specifically expressed in muscle cells, in particular skeletal muscle cells. In particular, the target gene or gene product is highly expressed in muscle cells compared to other cell types. The target genes or gene products include also genes and gene products from bacterial, fungal, parasitic and viral agents responsible for infectious myositis such as with no limitations Staphylococcus aureus, Candida spp., Trichinella spp., viruses such as Influenza A and B, and Enteroviruses such as Coxsackie.

The invention encompasses a pharmaceutical composition comprising two or more drugs associated to a syncytin protein, and/or a composition wherein at least two different syncytin proteins are associated to one or more drugs.

In the various embodiments of the present invention, the pharmaceutical composition, in particular the composition comprising particles as defined previously with syncytin displayed on their surface, and even more preferably lentiviral particles pseudotyped with syncytin packaging a drug of interest including a gene of interest, is used in any targeted therapy of muscle injuries or myopathies including regeneration phases as part of the disease physiopathological process by transducing cells of regenerating muscle tissue such as in particular myocytes, myotubes, myoblasts and/or satellite cells and more preferably myotubes, myoblasts and/or satellite cells.

Muscle cells (myocytes) are elongated cells ranging from several millimetres to about 10 centimetres in length and from 10 to 100 micrometres in width. These cells are joined together in tissues that may be either striated or smooth, depending on the presence or absence, respectively, of organized, regularly-repeated arrangements of myofibrillar contractile proteins called myofilaments. Striated muscle is further classified as either skeletal or cardiac muscle.

Skeletal muscle, which is attached to bones by tendons, is controlled by the peripheral nervous system and associated with the body's voluntary movements. Skeletal muscle is striated muscle. Skeletal muscle cells are covered by connective tissue, which protects and supports muscle fiber bundles. Blood vessels and nerves run through the connective tissue supplying muscle cells with oxygen and nerve impulses that allow for muscle contraction. In cardiac muscle cells are joined to one another by intercalated discs, which allow the synchronization of the heart beat. Cardiac muscle is branched, striated muscle. The heart wall consists of three layers: epicardium, myocardium, and endocardium. Myocardium is the middle muscular layer of the heart. Myocardial muscle fibers carry electrical impulses through the heart, which power cardiac conduction.

Visceral muscle (smooth muscle) is found in various parts of the body including blood vessels, the bladder, digestive tract, as well as in many other hollow organs. Like cardiac muscle, most visceral muscle is regulated by the autonomic nervous system and is under involuntary control. Visceral muscle has no cross striations. Visceral muscle contracts slower than skeletal muscle, but the contraction can be sustained over a longer period of time. Organs of the cardiovascular system, respiratory system, digestive system, and reproductive system are lined with smooth muscle.

Muscle regeneration after injury has similarities to muscle development during embryogenesis. Skeletal muscle repair is a highly synchronized process involving the activation of various cellular and molecular responses, where the coordination between inflammation and regeneration is crucial for the beneficial outcome of the repair process following muscle damage. Muscle tissue repair following damage can be considered as a process consisting of two interdependent phases: degeneration and regeneration, where, apart from the role of growth and differentiation factors, the degree of damage and the interactions between muscle and the infiltrating inflammatory cells appear to affect the successful outcome of the muscle repair process. Muscle regeneration depends on a balance between pro-inflammatory and anti-inflammatory factors that determine whether the damage will be resolved with muscle fiber replacement and reconstitution of a functional contractile apparatus, or with scar formation.

Following damage of the myofiber, quiescent satellite cells are activated to enter the cell cycle and proliferate, allowing for expansion of the myogenic cell population. At this stage, the satellite cells are called myogenic precursor cells. The proliferative phase is followed by the differentiation and fusion of myoblasts (differentiated satellite cells) with the damaged myofibers, for the repair of the fibers, or to each other, for new myofiber formation.

The myogenic cells that express Myf5 and MyoD are called myoblasts. Up-regulation of the secondary myogenic regulatory factors (MRFs) myogenin and MRF4 induces terminal differentiation of myoblasts into myocytes that now express not only myogenin and MRF4 but also important genes for muscle cells such as myosin heavy chain (MHC) and muscle creatine kinase (MCK). Eventually, mononucleated myocytes fuse to form multinucleated syncytium (myotubes), which finally mature into contracting muscle fibers (myocytes).

As satellite cell activation is not restricted to the damaged site, injury activates satellite cells all along the myofiber, leading to the proliferation and migration of satellite cells to the regeneration site.

Satellite cells (myogenic cells) are located within the basal lamina surrounding individual myofibers, between the plasma membrane of the muscle fiber and the basement membrane. In comparison to adult myofibers, they have unique morphological characteristics, including abundant cytoplasm, a small nucleus with increased amounts of heterochromatin and reduced organelle content. These features reflect the fact that satellite cells are mitotically quiescent and transcriptionally less active than myonuclei.

Skeletal muscle has the capacity for complete regeneration and repair after repeated injuries. This ability shows that the satellite cell pool is renewed after every regenerative process. It was however proposed that the self-renewal capacity of satellite cells is restricted. Thus, the exhaustion of the satellite cell pool after several rounds of regeneration may contribute to the clinical deterioration observed in the elderly or in patients with myopathies. For a detailed review on muscle regeneration see Karalaki M, Fili S, Philippou A, Koutsilieris M. Muscle regeneration: cellular and molecular events. In Vivo. 2009 September-October; 23(5):779-96; Baghdadi and Tajbakhsh 2018 (Meryem B Baghdadi, Shahragim Tajbakhsh. Regulation and phylogeny of skeletal muscle regeneration. Developmental Biology, Elsevier, 20172018)

The composition of the invention allows targeted delivery to the cells of the regenerating muscle tissue, in particular skeletal muscle tissue and/or cardiac muscle tissue. Typically, the composition allows targeted delivery to the cells of regenerating muscle tissue such as in particular myocytes, myotubes, myoblasts and/or satellite cells and more preferably myotubes, myoblasts and/or satellite cells.

As used ferein, the term “regenerating muscle tissue” refers to muscle tissue undergoing regeneration, i.e. myogenesis and new muscle formation.

In some embodiments of the invention, the pharmaceutical composition of the invention, in particular the composition comprising particles as defined previously with syncytin displayed on their surface, and even more preferably lentiviral vector particles pseudotyped with syncytin packaging a drug or gene of interest, preferably a gene of interest, is used for (targeted) gene therapy of muscle diseases.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed: this is gene editing therapy.

In additive gene therapy, the gene of interest may be a functional version of a gene, which is deficient or mutated in a patient, as is the case for example in a genetic disease. In such a case, the gene of interest will restore the expression of a functional gene. More preferably in such embodiment, the composition of the invention preferably comprises a viral vector coding for the gene of interest. Even more preferably, the viral vector is an integrative viral vector such as a retrovirus, notably a lentivirus as previously described.

Gene or genome editing uses one or more gene(s) of interest, such as: (i) a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); (ii) a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and eventually also a fragment of a functional version of a gene for use as recombination template, as defined above. Gene editing may be performed using non-integrative viral vectors such as non-integrative lentiviral vectors.

Of particular interest are deficient or mutated genes in patients exhibiting a muscle disease which once corrected in cells from the regenerating muscle tissue, improve the patient's disease or symptoms. The cells from the regenerating muscle tissue, in particular skeletal and/or cardiac muscle tissue, are preferably myocytes, myotubes, myoblasts, and/or satellite cells and more preferably myotubes, myoblasts, and/or satellite cells.

Muscle diseases according to the invention include but are not limited to the diseases as listed below.

Muscular diseases also named myopathies are diseases in which the muscle fibers do not function properly and which are generally associated with muscular damages. Myopathies according to the present invention include but are not limited to:
Dystrophies (or muscular dystrophies) including congenital muscular dystrophies are a subgroup of myopathies characterized by muscle degeneration and regeneration. Congenital muscular dystrophies (CMDs) distinguish themselves by the immunohistochemical finding of prominent dystrophic changes: muscle fiber necrosis and regeneration, increased endomysial connective tissue and replacement of muscle with fat tissue. Classical CMDs are clinically confined to the musculoskeletal system but other CMDs are characterized by significant cerebral neuronal migration defect and eye abnormalities. Dystrophies include:
   The dystrophinopathies, which include a spectrum of X-linked muscle diseases caused by pathogenic variants in DMD gene, which encodes the protein dystrophin. Dystrophinopathies comprises Duchenne muscular dystrophy, Becker muscular dystrophy (BMD) and DMD-associated dilated cardiomyopathy. DMD is the only gene in which pathogenic variants cause the dystrophinopathies. More than 5,000 pathogenic variants have been identified in persons with DMD or BMD. Disease-causing alleles are highly variable, including deletion of the entire gene, deletion or duplication of one or more exons, and small deletions, insertions, or single-base changes (see Darras B T, Miller D T, Urion D K. Dystrophinopathies. 2000 Sep. 5 [Updated 2014 Nov. 26]. In: Pagon R A, Adam M P, Ardinger H H, et al., editors. GeneReviews® [Internet]. Seattle (Wash.): University of Washington, Seattle; 1993-2017. Available from: https://www.ncbi.nlm.nih.gov/books/NBK1119/, as well as OMIM Entries for Dystrophinopathies 300376, 300377, 302045 and 310200).
   The Limb-girdle muscular dystrophies (LGMDs) which are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported.
      Autosomal dominant LGMDs (LGMD1) include:
         LGMD1A (myotilinopathy) caused by mutation of MYOT
         LGMD1B caused by mutation of LMNA. Pathogenic variants in LMNA result in at least eleven allelic conditions including LGMD1B, autosomal dominant and autosomal recessive Emery-Dreifuss muscular dystrophy, Dunnigan-type familial partial lipodystrophy (FPLD), mandibuloacral dysplasia, Hutchinson-Gilford progeria syndrome, and Charcot-Marie-Tooth type 2B1.
         LGMD1C (caveolinopathy) caused by mutation of the gene CAV3 encoding caveolin-3.
         LGMD1D, caused by mutation of DNAJB6 encoding for a protein being a member of the HSP/DNAJ family of molecular co-chaperones involved in protecting proteins from irreversible aggregation during protein synthesis or cellular stress.
         LGMD1E, caused by mutation in the desmin gene (DES).
         LGMD1F (TNPO3 gene), LGMD1G (HNRNPDL gene) and LGMD1H.
      Autosomal recessive LGMDs include:
         Sarcoglycanopathies, including α-sarcoglycanopathy (LGMD2D) caused by mutation of SGCA; β-sarcoglycanopathy (LGMD2E) caused by mutation of the gene SGCB; γ-sarcoglycanopathy (LGMD2C) caused by mutation of the gene SGCG; δ-sarcoglycanopathy (LGMD2F) caused by mutation of the gene SGCD.
         Calpainopathy (LGMD2A) caused by mutation of the gene CAPN3 with more than 450 pathogenic variants described.
         Dysferlinopathy (LGMD2B). Dysferlin (DYSF gene) is a sarcolemmal protein that includes C2 domains thought to be important for calcium-mediated vesicle fusion with sarcolemma and membrane repair of skeletal muscle fibers.
         LGMD2G involving TCAP pathogenic variants.
         LGMD2H involving pathogenic variants reported in TRIM32 including two missense variants, one codon deletion, and two frameshift variants.
         Dystroglycanopathies related to defects in O-linked glycosylation enzymes. including LGMD2I (caused by mutation of FKRP gene), LGMD2K (caused by mutation of POMT1 gene), LGMD2M (caused by mutation of FKTN), LGMD2O (caused by mutation of POMGNT1 gene), LGMD2N (caused by mutation of POMT2 gene).
         LGMD2L caused by defective variants of ANO5, encoding for actonamin a putative calcium-activated chloride channel possibly involved in membrane repair mechanism in muscular dystrophies.
         LGMD2J caused by defective variants of the TTN gene.
         LGMD2P caused by defective variants of the DAG1 gene
         LGMD2Q caused by defective variants of PLEC.
         LGMD2R caused by defective variants of DES.
         LGMD2S caused by defective variants of TRAPPC11.
         LGMD2T caused by defective variants of GMPPB.
         LGMD2U caused by defective variants of ISPD.
         LGMD2V caused by defective variants of GAA.
         LGMD2W caused by defective variants of LIMS2.
         LGMD2X caused by defective variants of BYES.
         LGMD2Y caused by defective variants of TOR1AlP1.
   The Emery-Dreifuss Muscular Dystrophy (EDMD) caused by defects in one of the gene including the EMD gene (coding for emerin), the FHL1 gene and the LMNA gene (encoding lamin A and C).
   Nesprin-1 and Nesprin-2 related muscular dystrophy caused by defects in the SYNE1 and SYNE2 gene, respectively; LUMA related muscular dystrophy caused by defects in the TMEM43 gene; LAP1B related muscular dystrophy caused by defects in the TOR1AIP1 gene.
   Facio-scapulo-humeral muscular dystrophy, type 1 (FSHD1A), such as associated with defect in the DUX4 gene (contraction of the D4Z4 macro satellite repeat in the subtelomeric region of chromosome 4q35) or the FRG1 gene; Facio-scapulo-humeral muscular dystrophy, type 2 (FSHD1B) caused by defects in the SMCHD1 gene.
   Muscular dystrophy with generalized lipodistrophy caused by defects in the PTRF gene.
   Muscular dystrophy with congenital disorder of glycosylation Type Io caused by defects in DPM3 gene.
   Scapuloperoneal muscular dystrophy and drop head syndrome caused by defects in VCP gene.
   Spinal muscular atrophies caused by pathogenic variants of SMN1 and/or SMN2.
   Oculopharyngeal muscular dystrophy (OPMD) caused by pathogenic variants of the gene PABPN1 encoding for the polyadenylate-binding nuclear protein 1.
   Congenital muscular dystrophies include Congenital muscular dystrophy with merosin deficient (LAMA2 gene); Bethlem myopathy (COL6A1, COL6A2, COL6A3, COL12A1 gene); Ullrich syndrome (COL6A1, COL6A2, COL6A3, COL12A1 genes) and other Congenital muscular dystrophies due to defects in the COL12A1, COL6A2, SEPN1, FHL1, ITGA7, DMM2, TCAP and LMNA genes; Congenital muscular dystrophies due to defective glycosylation (FKTN, POMPT1, POMPT2, FKRP, POMGNT1, POMGNT2, ISPD, B3GNT1, GMPPB, LARGE, DPM1, DMP2, ALG13, B3GALNT2, TMEM5, POMK genes); Other congenital muscular dystrophies (CHKB, ACTA1, TRAPPC11, GOLGA2, TRIP4 genes).
Congenital myopathies mostly characterized by muscle weakness related to reduced contractile ability of the muscles. Congenital myopathies include, but are not limited to:
   Nemaline myopathies characterized by the presence of “nemaline rods” in the muscle, and for which pathogenic variants in ten genes (NEB, ACTA1, TPM2, TPM3, TNNT1, CFL2, LMOD3, KBTBD13, KLHL40, and KLHL41) have been identified
   Core myopathies (central core myopathy, and multiminicore myopathy), characterized by multiple small “cores” or areas of disruption in the muscle fibers). Core myopathies are the most common form of congenital myopathy and are most commonly associated with RYR1 mutations. Mutations in the gene SEPN1 (encoding for SELENON) or in the gene encodin tropomyosin and in the KBTKD13 gene have also been observed in the multiple minicore and in the core-rod myopathies respectively. Mutations in the MEGF10 gene have also been disclosed in congenital myopathy with minicores.
   Congenital myopathy with fiber-type disproportion (MYH7 gene); Myopathy proximal to ophtalmoplegia (MYH2 gene); isolated inclusion body myopathy (HNRNPA1 gene); congenital skeletal myopathy and fatal cardiomyopathy (MYBPC3 gene); congenital lethal myopathy (CTCN1 gene; sarcotubular myopathy (TRIM32 gene); congenital myopathy related to PTPLA (PTPLA gene); congenital myopathy with ophtalmoplegia related to CACNA1S (CACNA1S gene).
   Centronuclear myopathy (or myotubular myopathy) associated with variants of the MTM1 (encoding for myotubularin), DNM2, BIN1, TNN, SPEG genes.
Distal myopathies associated with defects in the DYSF, TTN, GNE, MYH7, MATR3, TIA1, MYOT, NEB, CAV3, LDB3, ANO5, DNM2, KLHL9, FLNC, VCP, ADSSL1 genes.
Myofibrillar myopathies associated with defects in the CRYAB, DES, SEPN1, LDB3, MYOT, FLNC, BAGS, TRIM54, TRIM63, KY genes.
Miscellaneous myopathies associated with defects in the LAMP2, VMA21, CLN3, PABPN1, TNN, PLEC, MSTN, ACVR1, CAV3, FHL1, VCP, ISCU, RYR1, PYRODX1 genes.
Myotonic syndromes associated with defects in the DMPK, CNPB, CLCN1, CAV3, HSPG2, ATP2A1 genes; Myotonia include myotonia congenita, paramyotonia congenita and myotonic dystrophy.
Ion channel muscle diseases associated with defects in Chloride channel (CLCN1), Sodium channel (SCN4A, SCN5A), Calcium channel (CACNA1S, CACNA1A, Potassium channel (KCNE3, KCNA1, KCNJ18, KCNJ2, KCNH2, KCNQ1, KCNE2, KCNE1) genes. Example of Ion channel muscle disease is periodic paralysis.
Malignant hyperthermia associated with defects in RYR1, CACNA1S genes and other unknown genes.
Metabolic myopathies, which result from defects in biochemical metabolism that primarily affect muscle and include:
   Glycogen storage diseases such as

| | Defect in the gene coding | |
|---|---|---|
| Myopathy | for | Muscle symptoms |
| Glycogen storage | GYSI gene | Occasional muscle |
| disease Type 0 | (Glycogen synthase 3 | cramping |
| | Glycogen synthase 1 | |
| | (muscle) | |
| Glycogen storage | GAA gene | Muscle weakness |
| disease Type II | (acid alpha-glucosidase) | |
| (Pompe's disease) | | |
| Glycogen storage | GBE1 gene | Myopathy or |
| disease Type IV | (Glucan (1,4-alpha-), | Cardiomyopathy |
| | branching enzyme 1) | |
| Glycogen storage | AGL gene | Myopathy |
| disease Type IIIa | (amylo-1,6-glucosidase,4- | |
| (Cori's disease or | alpha-glucanotransferase) | |
| Forbes' disease) | Glycogen debrancher | |
| | enzyme | |
| Glycogen storage | PYGM gene | Exercise-induced |
| disease Type V | (Glycogen phosphorylase) | cramps, |
| (McArdle disease) | | Rhabdomyolysis |
| Glycogen storage | PKFM gene | Exercise-induced |
| disease Type VII | (phosphofructokinase | muscle cramps and |
| (Tarui's disease) | muscle) | weakness |
| Glycogen storage | PHKA1 gene | Exercise-induced |
| disease Type IXd | (phosphorylase b kinase | muscle weakness |
| (ex type VIII) or | alpha subunit) | and stiffness |
| muscle | | |
| phosphorylase | | |
| deficiency² | | |
| Congenital disorder | PGM1 gene | Exercise intolerance |
| of glycosylation | (Phosphoglucomutase) | and episodic |
| type It | | rhabdomyolysis |
| Glycogene | GYG1 gene | Muscle atrophy |
| storagedisease | (Glycogenin 1) | |
| type XV | | |
| Red cell aldolase | ALDOA gene | Exercise intolerance, |
| deficiency | Aldolase A | cramps |
| GSD type XIII | ENOS gene | Exercise intolerance, |
| | β-enolase | cramps |
| Glycogen storage | PRKAG2 gene | |
| disease of heart, | (Protein kinase, AMP- | |
| lethal congenital | activated, gamma 2 non- | |
| | catalytic subunit) | |
| Polyglucosan | RBCK1 gene | Myopathy |
| storage myopathy | (RanBP-type and C3HC4 | |
| | type zinc finger containing | |
| | 1 (heme-oxidized IRP2 | |
| | containing ubiquitin | |
| | ligase 1) | |

Diseases of the glycolytic pathway associated with defects in the PGK1, PGAM2, LDHA, ENO3 genes.
Disorders of lipid metabolism due to defects in the CPT2, SLC22A5, LC25A20, ETFA, ETFB, ETFDH, ACADVL, ACAD9, ABHD5, PNPLA2, LPIN1, PNPLA8 genes.
Congenital myasthenic syndromes associated with defects in GMPPB, MYO9A, SLC5A7, COL13A1, LRP4, PREPL, ALG14, ALG2, PLEC, SCN4A, LAMB2, DPAGT1, GFPT1, AGRN, DOK7, MUSK genes
Mitochondrial myopathies, which are due to defects in mitochondrial genes such as CHKB, MRPL3, NDUAF1, AARS2, MRPL44, MTO1, TSFM, CHCHD10, SLC25A42, PUS1, ADCK3, MARS2, MTPAP, YARS2, TK2 and SUCLA2. Examples include Kerans-Sayre syndrome (KSS), Leigh syndrome and maternally inherited Leigh syndrome (MILS), Mitochondrial DNA depletion syndrome, Mitochondrial encephalomyopathy, lactic acidosis and strokelike episodes (MELAS); Myoclonus epilepsy with ragged red fibers (MERFF), Neuropathy, ataxia and retinitis pigmentosa (NARP); Pearson syndrome and Progressive external ophtalmoplegia.
Lipid storage diseases including Niemann-Pick disease (types A, B, E, F: SMPD1 gene; types C, D: NPC1 gene), Fabry disease (GLA gene coding for alpha-galactosidase A), Krabbe disease (GALC gene), Gaucher disease (GBA gene), Tay-Sachs disease (HEXA gene), Metachromatic leukodystrophy (ARSA gene), multiple sulfatase deficiency (SUMF1 gene) and Farber disease (ASAH1 gene).
Hereditary cardiomyopathies associated with defects in the MYH6, MYH7, TNNT2, TPM1, MYBPC3, PRKAG2, TNNI3, MYL3, TTN, MYL2, ACTC1, CSRP3, TNNC1, VCL, MYLK2, CAV3, MYOZ2, JPH2, PLN, NEXN, ANKRD1, ACTN2, NDUAF1, TSFM, AARS2, MRPL3, COX15, MTO1, MRPL44, LMNA, LDB3, SCN5A, DES, EYA4, SGCD, TCAP, ABCC9, PLN, TMPO, PSEN2, CRYAB, FKTN, TAZ, DMD, LAMA4, ILK, MYPN, RBM20, SYNE1, MURC, DOLK, GATAD1, SDHA, GAA, DTNA, FLNA, TGFB3, RYR2, TMEM43, DSP, PKP2, DSG2, DSC2, JUP, CTNNA3, CASQ2, ANK2, KCNE1, KCNE2, KCNJ2, CACNAC1, SCN4B, AKAP9, SNTA1, KCNJ5, KCNH2, KCNQ1, NPPA, KCNA5, GJA5, SCN1B, SCN2B, NUP155, GPD1L, CACNA1, CACNB2, KCNE3, SCN3B, HCN4 genes.
Neuromuscular disorders caused by defects in the TOR1A, SGCE, IKBKAP, KIF21A, PHOX2A, TUBB3, TPM2, MYH3, TNNI2, TNNT3, SYNE1, MYH8, POLG, SLC25A4, C10orf2, POLG2, RRMB2, TK2, SUCLA2, SLC25A42, OPA1, STIM1, ORAI1, PUS1, CHCHD10, CASQ1, YARS2, FAM111B genes.
Neurogenic myopathies including the various types of Charcot-Marie-Tooth disease characterized by muscle atrophy and caused by mutations in various genes including DNM2, YARS, MP2, INF2, GNB4 and MTMR2, in particular Charcot-Marie-Tooth disease Type 4B1 due to defects in the MTMR2 gene; Amyotrophic Lateral Sclerosis (ALS)) characterized by muscle atrophy and caused by mutations in various genes including DCTN1, PRPH, SOD1 and NEFH.
Inflammatory myopathies, which are caused by problems with the immune system attacking components of the muscle, leading to signs of inflammation in the muscle. Inflammatory myopathies include autoimmune myopathies such as polymyositis, dermatomyositis, inclusion body myositis and myasthenia gravis.
Rhabdomyolysis, compartment syndrome or myoglobinuria. Rhabdomyolysisis a condition in which damaged skeletal muscle breaks down rapidly. Myoglobinuria Myoglobinuria is the presence of myoglobin in the urine, usually associated with rhabdomyolysis or muscle destruction. Trauma, vascular problems, malignant hyperthermia and certain drugs are example of situations that can destroy or damage the muscle, releasing myoglobin to the circulation. Other causes of myoglobinuria also include: McArdle's disease, Phosphofructokinase deficiency, Carnitine palmitoyltransferase II deficiency, Malignant hyperthermia, Polymyositis, Lactate dehydrogenase deficiency, Thermal or electrical burn.
Muscular necrosis (notably due to metabolic failure and/or membrane damage), sprains, distensions, cramps, tendonitis, contractures such as Volkmann's ischemic contracture or Dupuytren's contracture) muscle infections, myofascial pain and muscle twitching.

The muscle diseases according to the invention preferably include diseases involving muscle regeneration cycles such as, but not limited to, muscle dystrophies, rhabdomyolysis, muscular atrophy, muscular necrosis, and auto-immune myopathies such as for example myasthenia gravis and othermyopathies associated with muscle damage.

Muscle injuries include but are not limited to muscle damage produced by
a direct trauma;
physical exercise or overuse;
compartment syndrome;
drug abuse (such as alcoholic myopathy) or medication (such as glucocorticoid myopathy which lead to muscle atrophy) or exposure to myotoxic agents including radiations;
malignant hyperthermia;
ischemia;
exposure to hot or cold temperatures or electric burn.

Examples of mutated genes in genetic disease affecting the muscle as described above, include:
genes involved in muscular dystrophies such as DMD, MYOT, LMNA, CAV3, DES, DNAJB6, TNPO3, HNRNPDL, SGCA, SGCB, SGCG, SGCD, CAPN3, DYSF, TCAP, TRIM32, FKRP, POMT1, FKTN, POMGNT1, POMT2, ANO5, TTN, DAG1, DES, TRAPPC11, GMPPB, ISPD, GAA, LIMS2, BVES, TOR1AIP1,PLEC, EMD, FHL1, LMNA, SYNE1, SYNE2, TMEM43, DUX4, FRG1, SMCHD1, PTRF, DPM3, VCP, SMN1, SMN2, PABPN1, COL6A1, COL6A2, COL6A3, COL12A1, FHL1, ITGA7, DMM2, TCAP, LMNA, FKTN, POMPT1, POMPT2, FKRP, POMGNT1, POMGNT2, ISPD, B3GNT1, GMPPB, LARGE, DPM1, DMP2, ALG13, B3GALNT2, TMEM5, POMK, CHKB, ACTA1, TRAPPC11, GOLGA2 and TRIP4;
genes involved in congenital myopathies such as NEB, ACTA1, TPM2, TPM3, TNNT1, CFL2, LMOD3, KBTBD13, KLHL40, KLHL41, RYR1, SEPN1, KBTKD13, MTM1, MEGF10, MYH7, MYH2, HNRNPA1, MYBPC3, CTCN1 TRIM32 PTPLA, CACNA1S, MTM1, DNM2, BIN1, TNN and SPEG;
genes involved in Distal myopathies such as DYSF, TTN, GNE, MYH7, MATR3, TIA1, MYOT, NEB, CAV3, LDB3, ANO5, DNM2, KLHL9, FLNC, VCP and ADSSL1;
genes involved in Myofibrillar myopathies such as CRYAB, DES, SEPN1, LDB3, MYOT, FLNC, BAG3, TRIM54, TRIM63 and KY;
genes involved in Miscellaneous myopathies such as LAMP2, VMA21, CLN3, PABPN1, TNN, PLEC, MSTN, ACVR1, CAV3, FHL1, VCP, ISCU, RYR1 and PYRODX1;
genes involved in Myotonic syndromes such as DMPK, CNPB, CLCN1, CAV3, HSPG2 and ATP2A1;
genes involved in Ion channel muscle diseases such as CLCN1, SCN4A, SCN5A, CACNA1S, CACNA1A, KCNE3, KCNA1, KCNJ18, KCNJ2, KCNH2, KCNQ1, KCNE2 and KCNE1;
genes involved in Malignant hyperthermia such as RYR1 and CACNA1S;
genes involved in metabolic myopathies such as glycogen storage diseases: GYS1, GAA, GBE1, AGL, PYGM, PKFM, PHKA1, PGM1, GYG1, ALDOA, ENO3, PRKAG2 and RBCK1; Diseases of the glycolytic pathway: PGK1, PGAM2, LDHA and ENO3; Disorders of lipid metabolism such as CPT2, SLC22A5, LC25A20, ETFA, ETFB, ETFDH, ACADVL, ACAD9, ABHD5, PNPLA2, LPIN1 and PNPLA8;
genes involved in Mitochondrial myopathies such as CHKB, MRPL3, NDUAF1, AARS2, MRPL44, MTO1, TSFM, CHCHD10, SLC25A42, PUS1, ADCK3, MARS2, MTPAP, YARS2, TK2 and SUCLA2;
genes involved in Congenital myasthenic syndromes such as GMPPB, MYO9A, SLC5A7, COL13A1, LRP4, PREPL, ALG14, ALG2, PLEC, SCN4A, LAMB2, DPAGT1, GFPT1, AGRN, DOK7 and MUSK;
genes involved in Hereditary cardiomyopathies such as MYH6, MYH7, TNNT2, TPM1, MYBPC3, PRKAG2, TNNI3, MYL3, TTN, MYL2, ACTC1, CSRP3, TNNC1, VCL, MYLK2, CAV3, MYOZ2, JPH2, PLN, NEXN, ACTN2, NDUAF1, TSFM, AARS2, MRPL3, COX15, MTO1, MRPL44, LMNA, LDB3, DES, EYA4, SGCD, TCAP, ABCC9, PLN, TMPO, PSEN2, CRYAB, FKTN, TAZ, DMD, LAMA4, ILK, MYPN, RBM20, ANKRD1, SYNE1, MURC, DOLK, GATAD1, SDHA, GAA, DTNA, FLNA, TGFB3, RYR2, TMEM43, DSP, PKP2, DSG2, DSC2, JUP, CTNNA3, CASQ2, ANK2, KCNE1, KCNE2, KCNJ2, CACNAC1, SCN4B, AKAP9, SNTA1, KCNJ5, KCNH2, KCNQ1, NPPA, KCNA5-GJA5, SCN1B, SCN2B, NUP155, SCN5A, GPD1L, CACNA1, CACNB2, KCNE3, SCN3B and HCN4;
genes involved in Neuromuscular disorders such as TOR1A, SGCE, IKBKAP, KIF21A, PHOX2A, TUBB3, TPM2, MYH3, TNNI2, TNNT3, SYNE1, MYH8, POLG, SLC25A4, C10orf2, POLG2, RRMB2, TK2, SUCLA2, SLC25A42, OPA1, STIM1, ORAI1, PUS1, CHCHD10, CASQ1, YARS2 and FAM111B; and
genes involved in Neurogenic myopathies such as MTMR2, DNM2, YARS, MP2, INF2, GNB4 and MTMR2 (Charcot-Marie-Tooth diseases); DCTN1, PRPH, SOD1 and NEFH (Amyotrophic Lateral Sclerosis (ALS)).

Preferably, a gene of interest according to the invention is selected from genes, which are mostly, or specifically, expressed in the muscle include but not limited to the group comprising DMD, MYOT, CAV3, DES, SGCA, SGCB, SGCG, SGCD, CAPN3, DYSF, TCAP, POMT1, POMGNT1, POMT2, ANO5, FKTN, FKRP, TTN, EMD, FHL1, NEB, ACTA1, TPM2, TPM3, TNNT1, CFL2, LMOD3, KHL40, KHL41, RYR1, MTM1, SEPN1, DUX4, FRG1, MTMR2, the muscle glycogen phosphorylase (PYGM) and the muscle phosphofructokinase (PKFM).

Such genes may be targeted in the regenerating muscle tissue in replacement gene therapy, wherein the gene of interest is a functional version of the deficient or mutated gene.

Alternatively, these genes could be used as target for gene editing. A specific example of gene editing would be the treatment of Limb-girdle muscular dystrophy 2D (LGMD2D) which caused by mutations in the α-sarcoglycan gene (SGCA). The most frequently reported mutation, 229CGC>TGC (R77C) in exon 3 of SGCA, results in the substitution of arginine by cysteine. Thus, by gene editing a correct version of this gene in afflicted patients, this may contribute to effective therapies against this disease. Other genetic diseases of the muscle as listed above could be treated by gene editing using the same principle.

In gene therapy, it might be possible to use the composition of the invention as previously described and more particularly, the stable lentiviral particles pseudotyped with syncytin as per the invention in therapy for muscle tissue engineering, preferably endogenous muscle stem cells including satellite cells engineering, by transducing said cells (Nichols J E, Niles J A, Cortiella J. Design and development of tissue engineered muscle: Progress and challenges. Organogenesis. 2009, 5, 57-61).

It could also be possible to insert sequences favoring gene splicing, expression or regulation or gene editing. Tools such as CRISPR/Cas9 may be used for this purpose. This could be used to modify gene expression in cells of the regenerating muscle tissue, in the case of auto-immunity or cancer, or to perturb the cycle of viruses in such cells. In such cases, preferably, the heterologous gene of interest is chosen from those encoding guide RNA (gRNA), site-specific endonucleases (TALEN, meganucleases, zinc finger nucleases, Cas nuclease), DNA templates and RNAi components, such as shRNA and microRNA.

To treat infectious diseases of the muscle, the gene of interest may also target essential components of the muscle pathogen life cycle.

The pharmaceutical composition comprising stable pseudotyped lentiviral particles according to the invention could be used together or sequentially to target the same cells. This could be an advantage in strategies such as gene editing, in which multiple components of the gene editing platform need to be added to the cells.

In some other embodiments of the invention, the pharmaceutical composition of the invention comprising a drug associated to a syncytin protein, in particular the composition comprising particles as defined previously with syncytin displayed on their surface, and even more preferably lentiviral particles pseudotyped with syncytin packaging a drug or gene of interest, preferably a gene of interest, is used for immunomodulation or to modulate muscle transplant tolerance, notably in case of composite tissue allotransplantation which has been recently introduced as a potential clinical treatment for complex reconstructive procedures including traumatic injuries, cancer ablative surgeries, or extensive tissue loss secondary to burns. Composite tissue allografts (CTAs) consist of heterogeneous tissues including skin, fat, muscle, nerves, lymph nodes, bone, cartilage, ligaments, and bone marrow with different antigenicities. Thus, composite tissue structure is considered to be more immunogenic than solid organ transplants. Thus, the composition is administered to the transplant donor for the prevention of muscle transplant rejection. For these uses, the drug is in particular an immunosuppressive drug such as IL-10, CTLA4-Ig or other immunosuppressive peptides, or VEGF mutants that improve lymphangiogenesis (Cui et al. J. Clin. Invest. 2015, Nov. 2; 125(11):4255-68.) and the gene of interest is a gene encoding said immunosuppressive drugs or VEGF mutants.

In the various embodiments of the present invention, the pharmaceutical composition comprises a therapeutically effective amount of drug associated to syncytin protein.

In the context of the invention, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

Likewise, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with enveloped viruses and do not prevent virus entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS).

The invention provides also a method for treating a muscle disease, comprising: administering to a patient a therapeutically effective amount of the pharmaceutical composition as described above. The lower immunogenicity of LV pseudotyped with syncytin is expected to allow long-term gene expression in cells from regenerating muscle tissue by repeated administration of the pharmaceutical composition.

As used herein, the term “patient” or “individual” denotes a mammal. Preferably, a patient or individual according to the invention is a human.

The pharmaceutical composition of the present invention, in particular, the composition comprising particles as defined previously with syncytin displayed on their surface, and even more preferably lentiviral particles pseudotyped with syncytin packaging a drug of interest including a gene of interest, is generally administered according to known procedures, at dosages and for periods of time effective to induce a therapeutic effect in the patient.

The administration may be by injection, oral or local administration. The injection may be subcutaneous (SC), intramuscular (IM), intravenous (IV), intraperitoneal (IP), intradermal (ID) or else. Preferably, the administration is by injection. Preferably, the injection is intramuscular.

The invention relates also to a pharmaceutical composition for targeting regenerating muscle tissue, as defined above, comprising a drug of interest specific for muscular disease associated to syncytin protein, wherein the drug of interest including gene of interest, targets a gene or gene product (protein/peptide) involved in muscular disease(s) that is specifically, or mostly expressed in muscle cells, as defined above.

In some preferred embodiments, the pharmaceutical composition comprises a gene of interest for gene therapy of muscle diseases. Preferably, the gene of interest targets a gene responsible for a genetic disease affecting the muscle tissue, such as in particular selected from the group comprising: muscular dystrophies including dystrophinopathies, Limb-girdle muscular dystrophies, such as Sarcoglycanopathies, Calpainopathies and Dysferlinopathies, the Emery-Dreifuss Muscular Dystrophy, the Spinal muscular atrophy, the Oculopharyngeal muscular dystrophy, Nesprin-1, Nesprin-2 and LUMA related muscular dystrophy, Facio-Scapulo-Humeral Muscular Dystrophy (FSDH; type 1 and type 2), Muscular dystrophy with generalized lipodistrophy, Muscular dystrophy with congenital disorder of glycosylation Type Io, Scapuloperoneal muscular dystrophy and drop head syndrome and congenital muscular dystrophies; Distal myopathies; Myofibrillar myopathies; Miscellaneous myopathies; Myotonic syndromes; Ion channel muscle diseases such as familial periodic paralysis; Malignant hyperthermia; Congenital myopathies including nemaline myopathies, core myopathies and centronuclear myopathies; Mitochondrial myopathies; Metabolic myopathies including Glycogen storage diseases such as Pompe's disease, Cori's disease, Mc Ardle disease, Tarui's disease, Red cell aldolase deficiency, GSD type XIII, GSD type XV as well as lipid storages disease; Congenital myasthenic syndromes; neurogenic myopathies such as Charcot-Marie-Tooth diseases, for example Charcot-Marie-Tooth neuropathy Type 4B1 and Amyotrophic Lateral Sclerosis (ALS); lipid storage diseases; hereditary cardiomyopathies; neuromuscular disorders.

The target gene responsible for a muscle genetic disease can be selected from the group comprising DMD, MYOT, LMNA, CAV3, DES, DNAJB6, SGCA, SGCB, SGCG, SGCD, CAPN3, DYSF, TCAP, TRIM32, FKRP, POMT1, FKTN, POMGNT1, POMT2, ANO5, TTN, PLEC, EMD, FHL1, LMNA, SMN1, SMN2, PABPN1, NEB, ALTA1, TPM2, TPM3, TNNT1, CFL2, LMOD3, KBTBD13, KLHL40, KLHL41, RYR1, SEPN1, KBTKD13, MTM1, DUX4, FRG1 and MTMR2, the glycogen synthase gene (GYS1), the acid alpha-glucosidase gene (GAA), the glycogen debrancher enzyme (AGL), the muscle glycogen phosphorylase (PYGM), the muscle phosphofructokinase PKFM, the aldolase A gene (ALDOA), the β-enolase gene (ENO3), the glycogenin-1 (GYG1) gene and notably the target gene can be selected from the genes which are specifically or mostly expressed in the muscle, such as but not limited to DMD, MYOT, CAV3, DES, SGCA, SGCB, SGCG, SGCD, CAPN3, DYSF, TCAP, POMT1, POMGNT1, POMT2, ANO5, FKTN, FKRP, TTN, EMD, FHL1, NEB, ACTA1, TPM2, TPM3, TNNT1, CFL2, LMOD3, KHL40, KHL41, RYR1, MTM1, SEPN1, the muscle glycogen phosphorylase (PYGM), the muscle phosphofructokinase PKFM. The gene of interest is suitable for gene therapy of said genetic disease by gene replacement or gene editing, as defined above.

In some other preferred embodiments, the pharmaceutical composition comprises a gene of interest targeting an essential gene of a muscle pathogen. The pathogen can be selected from the group comprising Trichinella spp, enterovirus such as the Coxsackie virus, Influenza A and B viruses, Staphylococcus aureus, Candida spp and others (for review of the various muscle pathogens see notably Crum-Cianflone NF. Bacterial, Fungal, Parasitic, and Viral Myositis. Clinical Microbiology Reviews. 2008; 21(3):473-494).

In the various embodiments, the pharmaceutical composition preferably comprises particles with syncytin displayed on their surface, and even more preferably lentiviral particles pseudotyped with syncytin packaging a gene of interest for gene therapy of muscle diseases by targeting specifically a gene expressed in regenerating muscle tissue.

The practice of the present invention will employ, unless otherwise indicated, conventional techniques, which are within the skill of the art. Such techniques are explained fully in the literature.

In the various embodiments, viral particles, in particular viral vector particles, and virus-like particles may be produced using standard recombinant DNA technology techniques.

In particular, stable pseudotyped lentiviral particles including a heterologous gene of interest for use in the invention may be obtained by a method comprising the following steps:
a) transfecting at least one plasmid in appropriate cell lines, wherein said at least one plasmid comprises the heterologous gene of interest, the retroviral rev, gag and pol genes, and a nucleic acid coding for an ERV syncytin;
b) incubating the transfected cells obtained in a), so that they produce the stable lentiviral particles pseudotyped with an ERV syncytin, respectively, and packaging the heterologous gene of interest; and
c) harvesting and concentrating the stable lentiviral particles obtained in b).

The method allows obtaining high physical titers, as well as high infectious titers, of stable pseudotyped lentiviral particles including a heterologous gene of interest. Preferably, step c) of the method comprises harvesting, concentrating and/or purifying the stable lentiviral particles produced in step b), from the supernatant. Thus, preferably, the concentration of step c) comprises centrifugating and/or purifying the harvested stable lentiviral particles obtained in b). Said harvest may be performed according to well-known methods in the art. Preferably, the lentiviral vectors are harvested before fusion of the transfected cells, more preferably between 20 hours and 72 hours post-transfection, preferably after 24 hours. Preferably, the harvesting step consists of a single lentivirus harvest, preferably implemented between 20 and 72 hours post-transfection, preferably between 20 and 30 hours post-transfection, more preferably after 24 hours.

In step a), appropriate cell lines are transfected with at least one plasmid. Preferably, the transfection is a transient transfection. Preferably, appropriate cell lines are transfected with at least one, two, three or four plasmids. These cell types include any eukaryotic cell which support the lentivirus life cycle. Preferably, the appropriate cell lines are stable cell lines or cell lines refractory to the catastrophic consequences of the fusogenic effects of syncytins, so as to continue growing while producing the particles. Said appropriate cell lines are mammalian cell lines, preferably human cell lines. Representative examples of such cells include Human Embryonic Kidney (HEK) 293 cells and derivatives thereof, HEK293 T cells, as well as subsets of cells selected for their ability to grow as adherent cells, or adapted to grow in suspension under serum-free conditions. Such cells are highly transfectable.

The appropriate cell lines may already be expressing at least one, and at most four of the five sequences which are the heterologous gene of interest, the retroviral rev, gag and pol genes, and the nucleic acid coding for an ERV syncytin such as HERV-W, HERV-FRD or murine syncytinA, preferably in inducible form. In such a case, step a) comprises transfecting said cell line with at least one plasmid comprising at least one sequence which is not already expressed in said cell line. The plasmid mixture, or the single plasmid (if only one plasmid is used) is chosen such that, when transfected into said cell lines in step a), said cell lines express all five above sequences. For example, if the appropriate cell line expresses the retroviral rev, gag and pol genes, then the plasmid or mixture of plasmids to be transfected comprises the remaining sequences to be expressed, i.e. the heterologous gene of interest and the nucleic acid coding for an ERV syncytin such as HERV-W, HERV-FRD or murine syncytinA.

When one single plasmid is used, it comprises all the 5 sequences of interest, i.e.:
the heterologous gene of interest,
the rev, gag and pol genes, and
a nucleic acid coding for an ERV syncytin as previously described and notably coding for HERV-W, HERV-FRD or the murine syncytinA.

When two or three plasmids are used (plasmid mixture), each of them comprises some of the sequences of interest listed in the previous paragraph, so that the plasmid mixture comprises all the above cited sequences of interest.

Preferably four plasmids are used, and the quadritransfection comprises the following:
the first plasmid comprises the gene of interest,
the second plasmid comprises the rev gene,
the third plasmid comprises the gag and pol genes, and
the fourth plasmid comprises a nucleic acid coding for an ERV syncytin as previously described and notably coding for HERV-W, HERV-FRD or the murine syncytin-A.

Said quadritransfection is preferably performed with specific ratios between the four plasmids. The molar ratio between the different plasmids can be adapted for optimizing the scale-up of the production. The person skilled in the art is able to adapt this parameter to the specific plasmids he uses for producing the lentivirus of interest. In particular, the weight ratios of the first, second, third, fourth plasmids are preferably (0.8-1.2):(0.1-0.4); (0.5-0.8):(0.8-1.2), more preferably around 1:0.25; 0.65; 0.9.

The rev, gag and pol genes are retroviral, preferably lentiviral. Preferably, they are HIV genes, preferably HIV-1 genes, but could be also EIAV (Equine Infectious Anemia Virus), SIV (Simian immunodeficiency Virus), Foamy Virus, or MLV (Murine Leukemia Virus) virus genes.

The nucleic acid coding for the ERV syncytin, such as an ERV syncytin as previously defined and more preferentially coding for HERV-W, HERV-FRD or the murine syncytin-A is a DNA or cDNA sequence. Preferably, it corresponds to the cDNA sequence respectively listed in SEQ ID NO:1, 2 or 3, or to a sequence presenting at least 80%, preferably at least 90%, more preferably at least 95%, more preferably at least 99% identity with such SEQ ID NO:1, 2, or 3 respectively. Preferably, step a) comprises the transfection of at least the plasmid comprising, preferably consisting of, the cDNA sequence listed in SEQ ID NO:5 or 6. The term “identity” refers to the sequence similarity between two polypeptide molecules or between two nucleic acid molecule. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wis.) pileup program, or any of sequence comparison algorithms such as BLAST, FASTA or CLUSTALW.

The plasmids encoding the envelope glycoproteins which may be used are known to those skilled in the art such as the commercially available pCDNA3, backbone or any other plasmid cassette using a similar expression system, for instance using the CMV promoter such as the pKG plasmid described in Merten et al. (Human gene therapy, 2011, 22, 343-356).

According to step a), various techniques known in the art may be employed for introducing nucleic acid molecules into cells. Such techniques include chemical-facilitated transfection using compounds such as calcium phosphate, cationic lipids, cationic polymers, liposome-mediated transfection, such as cationic liposome like Lipofectamine (Lipofectamine 2000 or 3000), polyethyleneimine (PEI), non-chemical methods such as electroporation, particle bombardment or microinjection. The transfection of step a) is preferably carried out using calcium phosphate.

Typically, step a) may be performed by transient transfection of 293T cells with 4 plasmids (quadritransfection), in the presence of calcium phosphate. The 4 plasmids are preferably: a pKL plasmid expressing the HIV-1 gag and pol genes, a pK plasmid expressing HIV-1 rev gene, a pCCL plasmid expressing the heterologous gene of interest under control of a cellular promoter such as the human phosphoglycerate kinase (PGK) promoter and a pCDNA3 plasmid expressing an ERV syncytin, such as an ERV syncytin as previously defined and more preferentially expressing HERV-W (Syncytin-1), HERV-FRD (Syncytin-2) or the murine syncytin-A (Syncytin-A) or syncytin-B (syncytin-B) glycoproteins from a CMV promoter.

Then, after step a), the method comprises a step b) of incubating the transfected cells obtained in a), so that they produce, preferably in the supernatant, the lentiviral particles pseudotyped with an ERV syncytin, such as an ERV syncytin as previously defined and more preferentially pseudotyped with HERV-W, HERV-FRD or the murine syncytin-A including the heterologous gene of interest. Indeed, once step a) is performed, incubation of the obtained cells is performed. This leads to the production in the supernatant of the stable lentiviral particles, which are pseudotyped with an ERV syncytin, such as an ERV syncytin as previously defined and more preferentially pseudotyped with HERV-W, HERV-FRD or the murine syncytin-A and which include the heterologous gene of interest.

After transfection, the transfected cells are thus allowed to grow for a time which may be comprised between 20 and 72 hours post-transfection, in particular after 24 hours. The medium used for culturing the cells may be a classical medium, such as DMEM, comprising a sugar, such as glucose. Preferably, the medium is a serum-free medium. Culture may be carried out in a number of culture devices such as multistack systems or bioreactors adapted to the culture of cells in suspension. The bioreactor may be a single-use (disposable) or reusable bioreactor. The bioreactor may for example be selected from culture vessels or bags and tank reactors. Non-limiting representative bioreactors include a glass bioreactor (e.g. B-DCU® 2 L-10 L, Sartorius), a single-use bioreactor utilizing rocking motion agitation such as wave bioreactor (e.g. Cultibag RM® 10 L-25 L, Sartorius), single use stirrer tank bioreactor (Cultibag STR® 50 L, Sartorius), or stainless steel tank bioreactor.

After incubation, the obtained stable lentiviral particles are harvested and concentrated; this is step c). Preferably, the stable lentiviral particles obtained in b) are harvested before fusion of the transfected cells, more preferably 24h post-transfection. Preferably, the stable lentiviral particles present in the supernatant obtained in b) are centrifugated and/or purified. Said concentration step c) may be performed by any known method in the art, such as by centrifugation, ultrafiltration/diafiltration and/or chromatography.

The supernatant may be centrifugated at a speed comprised between 40000 and 60000 g, during 1h to 3h, at a temperature comprised between 1° C. and 5° C., so as to obtain a centrifugate of stable pseudotyped viral particles. Preferably, the centrifugation is performed at a speed of 45000 to 55000 g, during 1 h30 to 2 h30, at a temperature of 2° C. to 5° C., preferably around 4° C. At the end of this step, the particles are concentrated in the form of a centrifugate, which may be used.

Step c) may be chromatography, such as an anion exchange chromatography, or an affinity chromatography. The anion exchange chromatography may be preceded or followed by a step of ultrafiltration, in particular an ultrafiltration/diafiltration, including tangential flow filtration. The anion exchange chromatography is for example a weak anion exchange chromatography (including DEAE (D)-diethylaminoethyl, PI-polyethylenimine).

The invention will now be exemplified with the following examples, which are not limitative, with reference to the attached drawings in which:

### FIGURE LEGENDS

FIG. 1: Bioluminescent transgene expression in dystrophic mice (MDX) or in control mice (C57Bl/6) following intramuscular injection of LV-SynA or AAV2/8.

In each mouse the right Tibialis Anterior muscle (TA) was injected with 25 μL PBS and the left TA was injected with 25 μL of vector. In some mice the vector was 2.5.10¹¹ vector genome (vg) of rAAV8-Luc2 (AAV2/8 corresponding to AAV serotype 2 ITR and AAV serotype 8 capsid (C57BL/6 mouse on right of panel). In other mice 1.4.10¹¹ physical particles (pp) corresponding to 7.5.10⁵ infectious genomes (ig) of LV-SA-Luc2 was injected (LV-SynA; left panel C57BL/6 and middle panel mdx mice). Bioluminescence was measured 4 weeks post injection using the IVIS Lumina apparatus. A whole-body bioluminescence image of a representative mouse of each group is shown. Regions of interest (ROI) were defined manually and reported in each mouse to calculate signal intensities using the living image 3.2 software (Xenogen) and expressed as photons per second. Background photon flux was defined from an ROI drawn over the control mice in which no vector had been administered.

For each representative mouse, the bioluminescence signal-expressed as photons per second-in the right TA muscle (TA-R flux), corresponding to PBS control and in the left TA muscle (TA-L flux), corresponding to the vector is indicated.

FIG. 2: Immunohistological detection of the transgene expressed in muscle of MDX mice injected with LV-SynA vectors

Representative sections of a muscle of MDX mice injected with PBS (left panel) or LV-SA-Luc2 (LV-SynA-LucII; right panel). Both sections were stained with antibodies to luciferase and to laminin and with DAPI. Laminin staining shows the contour of myofibers and DAPI shows nuclei. Expression of luciferase is found in the cytoplasm of myofibers following LV-SA-Luc2 injection.

FIG. 3: Comparative bioluminescence obtained in skeletal muscle of mdx and C57BL/6 mice.

Seven to 8 mice per group having the right Tibialis Anterior muscle injected with 25 μL PBS and the left TA injected with 25 μL of LV-SA-Luc2 vector (between 1 to 1.4 10¹¹ physical particle/TA which corresponds to 0.75 to 1.10⁶ transducing unit (TU)/TA). Mdx mice were between 4.5 and 5.5 week old at the time of injection. C57BL/6 (B6) mice were between 6 and 8 week old at the time of injection. Bioluminescence in TA was measured one month after injection.

FIG. 4: Significant levels of transduction are obtained in muscles of MDX mice compared to normal mice, as determined by PCR and by quantification of vector copy number in injected TA using qPCR.

In each mouse the right Tibialis Anterior muscle (TA) was injected with 25 μL of PBS and the left TA was injected with 25 μL of vector LVSynA (LVSynA-Luc), corresponding 5.10⁵ infectious genomes (ig). DNA samples were analysed at 4 and 6 weeks post-vector injection by q-PCR (A, C) and by PCR (B, D). Levels of vector copy number per diploid nucleus (VCN) were quantified by amplifying Psi vector sequences normalized to the murine titin gene levels using qPCR. In A and C, boxes represents the averaged VCN values obtained for all mice and lines show the minimum and maximum values obtained. In B and D, the 489 bp band corresponding to the integrated vector is detected only in the MDX mice muscles injected with the LVSynA vector. No band at 489 bp detected in the control muscles injected with PBS. Data are representative of 12 mice per condition. Comparisons between PBS and LVSynA groups were performed using a Mann and Whitney two-tailed analysis. P value under 0.05 was considered statistically-significant.

FIG. 5: Gene transfer in sgca−/− mice muscle with a LV pseudotyped with syncytin A.

Six-week old sgca−/− mice (n=2) were injected with 10⁶ ig of LV-SynA Luc2 vector per TA in 25 μL volume. The left TA was injected with the luciferase-coding vector and the right TA was injected with a control vector. Bioluminescence was measured 14 days following vector injection. A whole-body bioluminescence image of one representative mouse out of two is shown. Regions of interest (ROIs) were defined manually and reported in each mouse to calculate signal intensities using the living image 3.2 software (Xenogen) and expressed as photons per second. Background photon flux was defined from an ROI drawn over the control mice in which no vector had been administered. A signal of 8.5 10³ photons/sec was detected in the left muscle injected with PBS, and a signal of 1.96 10⁶ photons/sec was detected in the right muscle injected with the LV-SA Luc2 vector.

FIG. 6: Significant transduction of another dystrophic model, sgca-deficient mice with LV-SynA vectors as shown by quantification measure of vector copy number in injected TA by q-PCR and detection of the vector copy number in injected TA using qPCR.

In each 6 week-old sgca−/− mice the right Tibialis Anterior muscle (TA) was injected with 25 μL of PBS and the left TA was injected with 25 μL of vector LVSynA (LVSynA-Luc), corresponding 5.10⁵ infectious genomes (ig). DNA samples were analysed at 4 weeks post-vector injection by q-PCR (A) and by PCR (B). Levels of VCN in the TA were measured by q-PCR and normalized to Titin levels. In (A) boxes and lines represent the VCN values obtained for each mice with the mean and the min and the max values obtained. In (B) the 489 bp band corresponding to the integrated vector is detected in the sgca deficient mice muscles injected with the LVSynA vector. No band at 489 bp detected in the control muscles injected with PBS. Data are representative of at least 11 mice per condition. Comparisons between PBS and LVSynA groups were performed using a Mann and Whitney two-tailed analysis. P value under 0.05 was considered statistically-significant.

FIG. 7: Detection of exon 23-skipped dystrophin mRNA.

Mdx mice were injected intramuscularly (IM) with lentiviral vector pseudotyped with syncytin A and coding for the mex23 antisense sequence expressed from the U7 promoter (LV-SA U7mex23) or with AAV1 vector coding for the U7-driven antisense mex23 sequence (rAAV U7mex23). RNA samples were analysed at 2 weeks post-vector injection by nested RT-PCR with primers in exons 20 and 26. The 901 bp band corresponding to the full-length dystrophin mRNA, is detected in all muscles, and the 688 bp fragment corresponding to the exon 23-skipped mRNA detected only in the muscles injected with the AAV vector (lanes 4, 5 and 6) or with Lv-SynA vector (lanes 1, 2 and 3). No band at 688 bp detected in the control muscles injected with PBS or with a vector coding for Luc2.

FIG. 8: Stable transduction of MDX mice is obtained with LV SynA contrary to LVVsvg, as determined by bioluminescence signal kinetics.

The right Tibialis Anterior muscle (R-TA) of MDX and C57BL/6 mice was injected with 25 μL of PBS and the left TA (L-TA) was injected with 25 μL of LVSynA (LV-SYNA LUC2) or LVVsvg (LV-VSVg LUC2) expressing the luciferase (Luc2) transgene and corresponding to the injection of 5.10⁵ infectious genomes (IG) per mouse. Bioluminescence was measured in the R-TA and L-TA at the indicated time points. Quantification was performed with the Ivis Lumina using the Living.Image 3.3 software. The dotted line is the quantification limit area (not the detection limit). Data represent 3 independent experiments in C57Bl/6 mice and 5 independent experiments in MDX mice, each including at least 3 mice per group for LVSynA conditions, and 1 experiment with 4 mice per group for LVVsvg conditions.

FIG. 9 Stability of transgene expression following LV-SynA intramuscular delivery in animal models of muscular dystrophies as shown by bioluminescence kinetics.

The right Tibialis Anterior muscle (R-TA, black line)) of Sgca deficient and MDX mice was injected with 25 μL PBS and the left TA (L-TA, grey line) was injected with 25 μL of LVSynA encoding Luc2 (LVSynALuc2), a dose corresponding to 2 to 7.5.10⁵ infectious genomes (ig). Bioluminescence was measured in the R-TA and L-TA at the indicated time points. Quantification was performed with the Ivis Lumina using the Living. Image 3.3 software. Data represent three independent experiments in Sgca deficient mice and five independent experiments in MDX mice, each including at least 3 mice per group.

FIG. 10: Reduced immune responses in an animal model of muscular dystrophy following intramuscular (IM) injection of LVSynA compared to LVVsvg as determined by Elispot anti-IFNg, PCR, q-PCR and immunohistochemistry.

The GFP-HY transgene is a model used to detect anti-transgene CD4 and CD8 T cell immune responses. The GFP-HY transgene encodes a fusion protein composed of the fluorescent protein GFP tagged with the HY male polypeptide. Following gene transfer, antigenic presentation of the transgene product can be specifically detected by Dby and Uty peptide presentation to CD4 and CD8 T cells respectively.

Four week-old MDX mice were injected IM into the TA with PBS, 5.10⁹ physical particles of LVSynA_GFP-HY or LVVsvg_GFP-HY vectors.
(A) Fourteen days later, spleen cells were harvested to measure Dby-specific CD4+T cell and Uty-specific CD8+ T cell response by γIFN-ELISPOT following peptide in vitro stimulation. Data represent one experiment including 3 mice per group.
(B) and (C) Muscle DNA samples were analysed at 2 weeks post-vector injection by PCR (B) and by q-PCR (C). In (B) the 489 bp band corresponding to the integrated vector is detected in the MDX mice muscles injected with either of the vectors but appears to be stronger in muscles injected with LVSynA compared to LVVsvg. In (C) levels of VCN in the TA were measured by q-PCR and normalized to Titin levels. Levels were higher in muscles injected with LVSynA compared to LVVsvg.
(D) Immunohistological analysis of CD3 expression was performed on cryosections of MDX muscles injected with the indicated vectors, before staining of the nuclei with Dapi. Each nucleus was then segmented and counted based on dapi staining intensity (empty grey circle) with the image j software. CD3 signal intensity was quantified in each nucleus to determine the distribution and the percentage of CD3 positive nuclei on the muscle section (full back circle). Images are representative of 3 muscle cross-sections per group with 3 mice analyzed per group.

FIG. 11: Reduced immune response against transgene following systemic delivery using LVSynA, compared to LVVsvg, as measured using Elispot anti-IFNg and CBA.

Six-week-old C57BL/6 mice were injected IV into the tail vein with PBS, 7.5.10⁵ Ig/mouse of LVSynA_GFP-HY or LVVsvg_GFP-HY vectors.
(A) Twenty-one days later, spleen cells were harvested to measure Dby-specific CD4+T cell and Uty-specific CD8+ T cell response by γIFN-ELISPOT following peptide in vitro stimulation. Data represent one experiment including 3 mice per group. For the titration of cytokines secreted by T cells.
(B) Three weeks after the immunization, total splenocytes were re-stimulated in vitro by Dby, Uty peptides, or Concanavalin A (conA) as positive control. After 36 h of culture, supernatants were removed and titrated for the indicated cytokines (3 mice/group/experiment). Each point represents an individual measurement with at least 2 measurement per mice.

FIG. 12: In vivo correction of gene deficiency of sgca-deficient mice is feasible by gene transfer with Lv-SynA Sgca vector and the expression of the therapeutic transgene can be enhanced by repeated injections of vector in the same muscle.

In each Sgca-deficient mouse, the right Tibialis Anterior muscle (TA) was injected with 25 μL PBS and the left TA was injected one time or two times with 25 μL of vector LVSynA (LVSynA-PGK-halpha-sarcoglycan), corresponding 2.5.10⁵ infectious genomes (ig) per TA. DNA and RNA samples were analysed at 16 days post-vector injection.
(A) The 489 bp band corresponding to the integrated vector is detected in the sgca deficient mice muscles injected one time and two times with the LVSynA vector. No band at 489 bp detected in the control muscles injected with PBS.
(B) The expression level of alpha-sarcoglycan mRNA was measured by qRT-PCR and normalized to P0 levels.
(C) Vector genome copies in the TA were measured by q-PCR and normalized to Titin in both cases (HIV and AAV).

FIG. 13: Transduction of regenerating muscle cannot be predicted from in vitro data as shown by in vitro transduction of C2C12 cells at different stages, with Lv-Syn vectors.

C2C12 murine myoblasts cell line were cultured (A) in growth medium (DMEM+10% FCS+1% Glutamine+1% PS) and transduced with the indicated LV syncytins (1×E+05 IG/mL) or LV VSVg (1^{E}+06 IG/mL) in the presence of Vectofusin-1 (12 μg/mL). The vectors used were LVSynA-ΔNGFR, LVSynB-ΔNGFR, LVSyn1-ΔNGFR, LVSyn2-ΔNGFR, LVVsvg-ΔNGFR. The percentage of transgene-expressing cells were measured by flow cytometry using the LSRII device and analysed with Diva software at day 7 and data were averaged from 3 experiments. In (B), C2C12 cells were induced to differentiate by changing the medium and culturing them in differentiation medium (DMEM+2% Horse serum+1% Glutamine+1 PS). At different times, cells were transduced with increasing volumes of the indicated vectors, either immediately (d0) or 1 or 3 days (d1 or d3) after medium change. Transgene expression was measured after 3 days, using flow cytometry on the LSRII device with Diva software analysis. Data are representative of 2 different experiments.

FIG. 14: Comparison between the level of expression of mLy6e mRNA and the level of transduction on different cell lines.
(A) mRNA were extracted from different cell lines (A20IIA, C2C12, NIH/3T3) and converted into cDNA to perform a qRT-PCR on mLy6e, using PO as a housekeeping gene. Relative levels were calculated with the formula abundance=2^{-ΔCt}. The qRT-PCR was validated by testing total cells from the lung, spleen or bone marrow of C57BL/6 mice which confirmed that the mLy6e expression level was the highest in lung cells, as published by Bacquin et al, J. Virol., 2017, doi: 10.1128/JVI.00832-17.
(B) The same cell lines as in FIG. 14 (A) were transduced with LV-Syncytin A vectors encoding ΔNGFR at a dose of 10⁵ IG/mL. The level of transduction was analysed by flow cytometry at 7 days post-transduction.

FIG. 15: In vitro transduction of human skeletal muscle myoblasts cells with human Syncytin 2 LV vectors.

Primary CSC-C3196 human skeletal muscle myoblasts cells which were obtained from a post-natal human muscle and purchased from the Creative Bioarray company, were transduced with 5.10⁵ ig/mL of LVSyncytin vectors comprising: LVSyn2-GFP and LVSynB-GFP in the presence of vectofusin. After 5 days cultured, the number of transduced cells (GFP positive cells) and the HIV vector copy number was observed by microscopy (A) using the EVOS FL device (Life Technologies) and by q-PCR (B) respectively. Each field is representative of two culture wells. Data are representative of 2 different experiments. Magnification 10×.

### EXAMPLE 1: PRODUCTION OF STABLE AND INFECTIOUS LV-SYNA PARTICLES

Materials and Methods

Cell Lines

Human embryonic kidney 293T cells were cultured at 37° C., 5% CO2 in Dulbecco's modified Eagle's medium (DMEM+glutamax) (Life Technologies, St-Aubin, France) supplemented with 10% of heat inactivated fetal calf serum (FCS) (Life Technologies).

Cloning of Syncytin A and Production of LV-Syn A.

a. Generation of a Plasmid Expressing Murine Syncytin-A.

Murine syncytin-A cDNA was cloned into a pCDNA3 plasmid using standard techniques.

b. Production of Syn-A-Pseudotyped Lentiviral Vectors.

HEK293T cells were co-transfected with the following 4 plasmids (quantities per flask), using calcium phosphate: pKLgagpol expressing the HIV-1 gagpol gene (14.6 μg), pKRev expressing HIV-1 rev sequences (5.6 μg), pcDNA3.1SynA (20 μg), and gene transfer plasmid (22.5 μg). LV-SA-Luc2 was produced using gene transfer plasmid PRRL-SFFV LucII, expressing Luciferase 2 transgene under control of the Spleen Focus Forming Virus (SFFV) promoter. LV-SA U7mex23 was produced using gene transfer vector coding for the mex23 antisense sequence under control of U7 promoter obtained from a previously described construct (Goyenvalle et al. Science, 2004, 3; 306(5702):1796-9). After 24 hours, the cells were washed and fresh medium was added. The following day, medium was harvested, clarified by centrifugation 1500 rpm for 5 min and filtered 0.45 μm, then concentrated by ultracentrifugation 50000 g for 2h at 12° C. and stored at −80° C. until used.

c. Titration of Syncytin-A-pseudoptyped LV

Physical titer was determined by p24 ELISA (Alliance© HIV-1 Elisa kit, Perkin-Elmer, Villebon/Yvette, France) followed by a calculation of the titer as physical particles (pp) assuming that 1 fg of p24 corresponds to 12 pp of LV (Farson et al, Hum Gene Ther. 2001, 20, 981-97), as previously reported for other types of LV (Charrier et al, Gene therapy, 2011, 18, 479-487). Infectious titer was determined as infectious genome titer (IG/mL) using the murine lymphoma cell line A20. Serial dilutions of vector are added to A20 cells in the presence of Vectofusin-1® (12 μg/μL) for 6 hours. Medium is renewed and cells are incubated for 7 days and genomic DNA is obtained to measure vector copy number per cells using duplex qPCR on iCycler 7900HT (Applied Biosystems) with the primers: PSI forward 5′CAGGACTCGGCTTGCTGAAG3′ (SEQ ID NO:7), PSI reverse 5′TCCCCCGCTTAATACTGACG3′ (SEQ ID NO:8), and a PSI probe labeled with FAM (6-carboxyfluoresceine) 5′CGCACGGCAAGAGGCGAGG3′ (SEQ ID NO:9), Titin forward 5′AAAACGAGCAGTGACGTGAGC3′ (SEQ ID NO:10), Titin reverse 5′TTCAGTCATGCTGCTAGCGC3′ (SEQ ID NO:11) and a Titin probe labeled with VIC 5′TGCACGGAAGCGTCTCGTCTCAGTC3′ (SEQ ID NO:12).

Results

Murine Syncytins were explored as possible new pseudotype for HIV-1-derived LV for in vivo applications. Syncytin A is non-orthologue but functionally similar murine counterpart to human Syncytins-1 and -2 (Dupressoir et al, Proceedings of the National Academy of Sciences of the United States of America, 2005, 102, 725-730).

The murine SynA was cloned into an expression plasmid and used to produce lentiviral vector particles in 293T cells. It was found that SyncytinA can successfully pseudotype rHIV-derived LV. An optimization of the amount of SyncytinA plasmid for the transfection step increased the production of LV particles based on p24 levels in medium. In the conditions defined (20 μg DNA per plate, one harvest only; see Materials and Methods), it was possible to produce stable and infectious particles pseudotyped with murine syncytin. Lentiviral particles pseudotyped with this envelope could be successfully concentrated by ultracentrifugation using the same conditions as used for VSVg-pseudotyped particles (Charrier et al, Gene therapy, 2011, 18, 479-487). The concentrated stocks were cryopreserved at −80° C. and were stable for several months. LV-Syn A was very efficient at transducing the murine A20 B lymphoma cell line in the presence of Vectofusin-1 (VF1). The A20 cell line is used to generate the infectious titer for Syncytin-A-pseudotyped LV.

### EXAMPLE 2: IN VIVO GENE DELIVERY TO REGENERATING SKELETAL MUSCLE USING LV-SYNA PARTICLES

Materials and Methods

Animals

Male C57/B16 mice aged 6-8 weeks were used for experiments and were purchased from Charles River. Male mdx mice aged 4-5.5 weeks were obtained from the Genethon breeding colony. Six week old sgca−/− mice deficient in alpha sarcoglycan were obtained from the Genethon breeding colony. Mice were injected in the tibialis anterior muscle (TA) using a 25 μL volume. Mice injected with luciferase vectors were analyzed by bioluminescence at different time points and sacrificed. Mice injected with small nuclear RNA mex23 expressing vectors were sacrificed for analysis. Right and Left Tibialis anterior (TA) muscles were removed after sacrifice. qPCR and RT-PCR were performed on part of frozen TA muscles. To perform microtome slices and immunohistostaining, the other part of TA muscles were fixed and embedded in paraffin.

In Vivo Luciferase Imaging

C57BL/6 mice were anesthetized with ketamine (120 mg/kg) and xylasine (10 mg/kg) and 100 μL (150 μg/mL) of D-luciferin (Interchim, ref FP-M1224D) was administered intra-peritoneally and imaged 10 min later with a CCD camera ISO14N4191 (IVIS Lumina, Xenogen, Mass., USA). A 3 min bioluminescent image was obtained using 10 cm field-of-view, binning (resolution) factor 4, 1/f stop and open filter. Region of interest (ROIs) were defined manually (using a standard area in each case), signal intensities were calculated using the living image 3.2 software (Xenogen) and expressed as photons per second. Background photon flux was defined from an ROI drawn over the Right Tibialis anterior muscle (TA-R) in which no vector had been administered.

qPCR

Genomic DNA is extracted from the cells using the Wizard® Genomic DNA Purification Kit (Promega, ref A1125). The multiplex qPCR is performed either on the PSI proviral sequence or on the WPRE proviral sequence, with the TitinMex5 as a normalization gene. The following primers and probes are used at a concentration of 0.1 μM:

| | |
|---|---|
| PSI F | 5′ CAGGACTCGGCTTGCTGAAG 3′ |
| | (SEQ ID NO: 7) |
| PSI R | 5′ TCCCCCGCTTAATACTGACG 5′ |
| | (SEQ ID NO: 8) |
| PSI probe (FAM) | 5′ CGCACGGCAAGAGGCGAGG 3′ |
| | (SEQ ID NO: 9) |
| WPRE F | 5′ GGCACTGACAATTCCGTGGT 3′ |
| | (SEQ ID NO: 13) |
| WPRE R | 5′ AGGGACGTAGCAGAAGGACG 3′ |
| | (SEQ ID NO: 14) |
| WPRE probe (FAM) | 5′ ACGTCCTTTCCATGGCTGCTCGC 3′ |
| | (SEQ ID NO: 15) |
| TitinMex5 F | 5′ AAAACGAGCAGTGACGTGAGC 3′ |
| | (SEQ ID NO: 10) |
| TitinMex5 R | 5′ TTCAGTCATGCTGCTAGCGC 3′ |
| | (SEQ ID NO: 11) |
| TitinMex5 probe | 5′ TGCACGGAAGCGTCTCGTCTCAGTC 3′ |
| (VIC) | (SEQ ID NO 12) |

The qPCR mix used is ABsolute qPCR ROX mix (Thermo Scientific, ref CM-205/A). The analysis is performed on the iCycler 7900HT (Applied Biosystems) with the SDS 2.4 software.

PCR

PCR on integrated lentiviral vector was performed using the following primers at a concentration of 0.1 μM: Psi-F: AGCCTCAATAAAGCTTGCC (SEQ ID NO: 20) and RRE-R:TCTGATCCTGTCGTAAGGG (SEQ ID NO: 21).

Immunohistostaining on Muscle Sections

Mice muscle are fixed in formalin solution with 10% formaldehyde (VWR) during at least 2 hours before being embedded in paraffin. Microtome sections of muscle (4 μm) are then stained with a polyclonal antibody anti-luciferase (Promega, ref G7451) diluted at 1/100 as a primary antibody and a donkey anti-goat AlexaFluor 594 (Invitrogen, ref A11058) diluted at 1/1000 as a secondary antibody. The primary antibody is incubated overnight at 4° C. in a humidity chamber and the secondary antibody is incubated for 2h in a humidity chamber.

Detection of Dystrophin Exon Skipping

Total RNA was isolated from pooled intermediate muscle sections using TRIzol-reagent (Life Technologies). To detect dystrophin mRNA, nested RT-PCR was carried out with 200 ng of total RNA using access RT-PCR system (Promega). The first reaction was performed with Ex20ext (5′-CAGAATTCTGCCAATTGATGAG-3′, SEQ ID NO: 16) and Ex26ext (5′-TTCTTCAGCTTGTGTCATCC-3′, SEQ ID NO: 17) primers for 30 cycles (94° C./30 s; 55° C./1 min; 72° C. 2 min). Then 2 μL of the first reaction were amplified for 23 cycles with Ex20int (5′-CCCAGTCTACCACCCTATCAGAGC-3′, SEQ ID NO: 18) and Ex26int (5′ CCTGGCTTTAAGGCTTCCTT-3′, SEQ ID NO: 19). PCR products were analysed on 2% agarose gel

Statistical Analysis (Luciferase and qPCR Experiments)

In the overall study, comparisons between 2 experimental groups were performed using a Mann and Whitney two-tailed analysis. P value under 0.05 was considered statistically-significant.

Results

The objective was to test if LV pseudotyped with Syncytin A could enter into myofibers of regenerating muscles but not in steady-state normal muscle. Therefore, young mdx (or MDX) mice (less than 12 weeks) deficient in dystrophin, a model of Duchenne Muscular Dystrophy, which are known to be in constant regenerative phase in their muscle deficient in dystrophin were used. Sarcoglycan-deficient mice which are undergoing muscle regeneration were also used. The murine syncytin-A glycoprotein was used to pseudotype HIV-1-derived lentiviral vectors encoding several transgene sequences: either the luciferase LucII (or Luc2) to facilitate the detection of transgene expression by bioluminescence, or a small antisense sequence for dystrophin exon 23 skipping (U7mex23) to show a functional effect.

LV-SynA vectors coding for LucII were injected intramuscularly into the tibialis anterior (TA) of male mdx mice or C57BL/6 albinos controls. As a positive control, the same volume of a rAAV2/8 (AAV2 genome packaged in AAV8 capsid) coding for LucII was injected using the same route. Two protocols have been performed to examine the bioluminescence obtained in the mice over time following the IM injections.

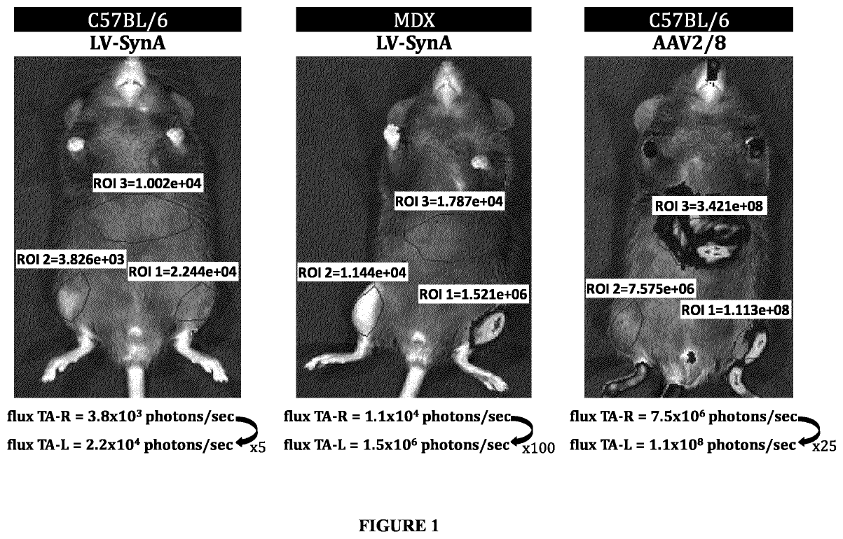

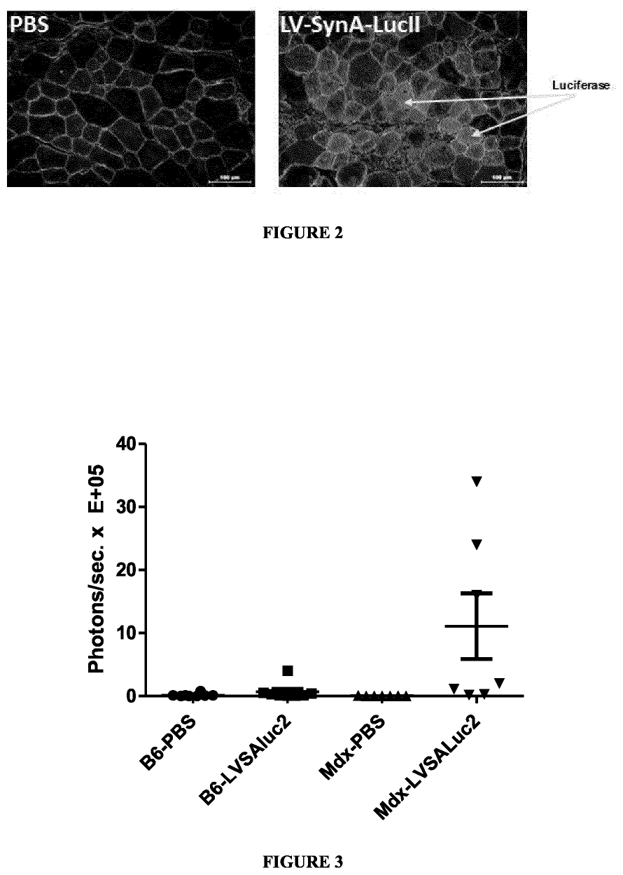

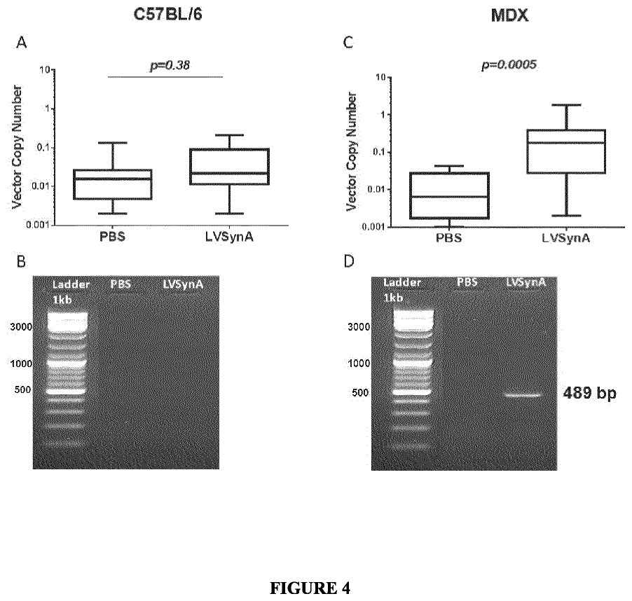

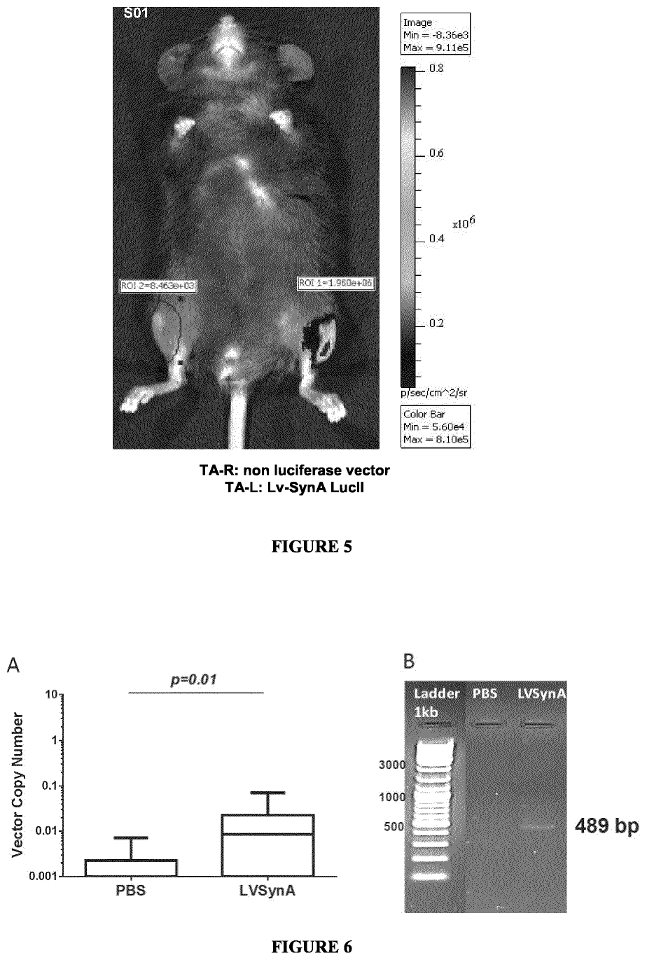

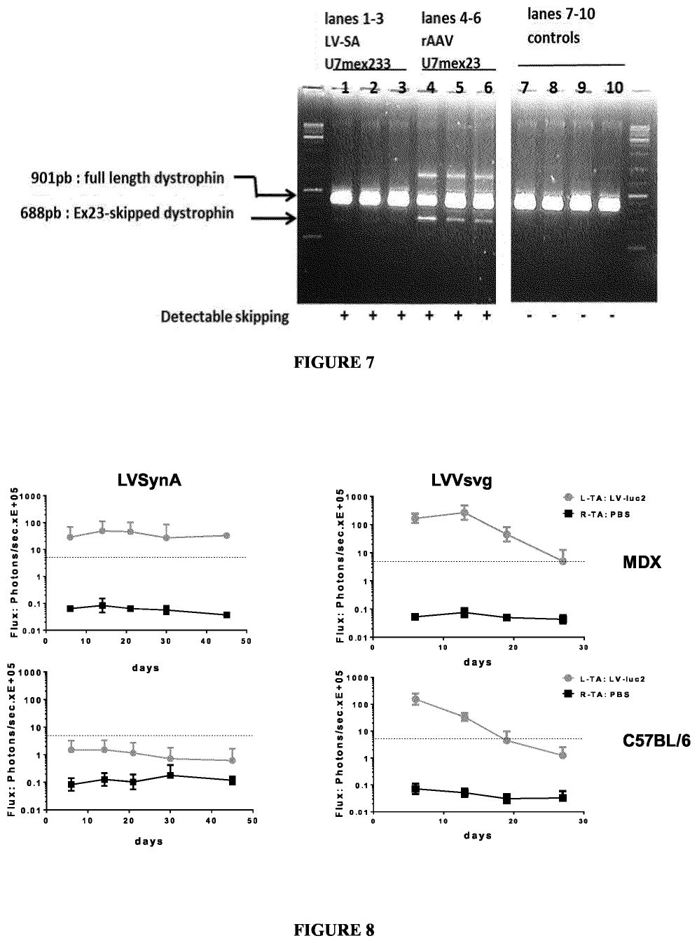

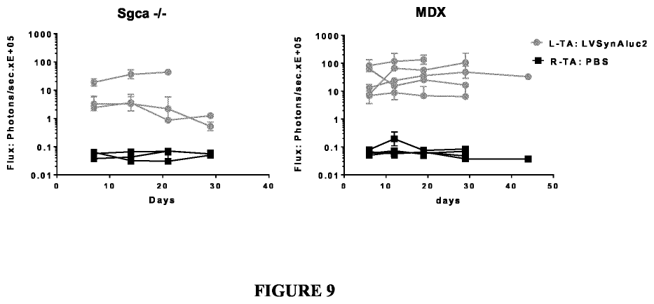

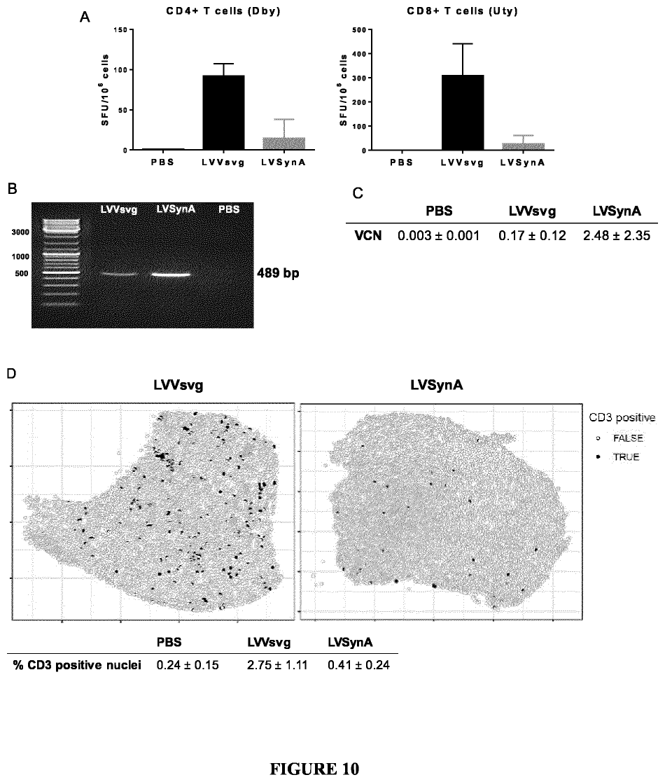

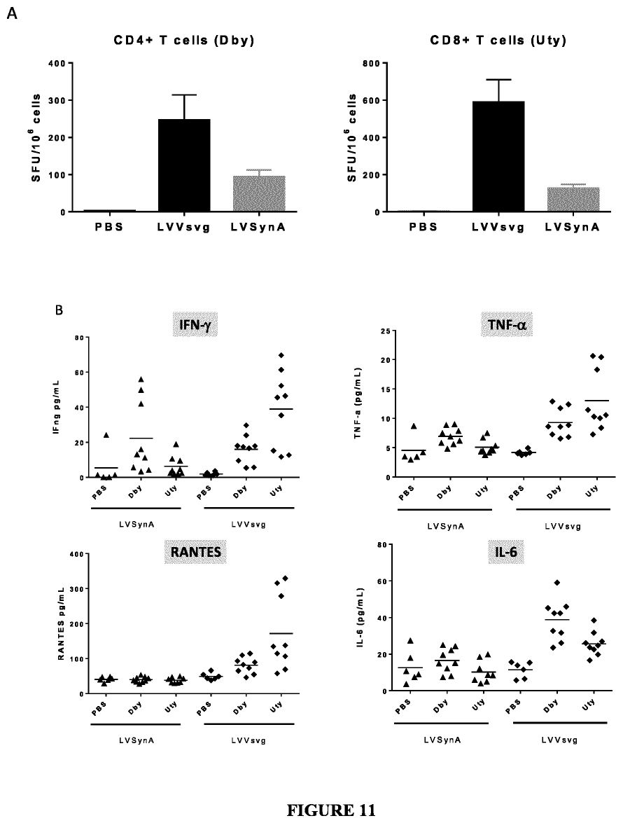

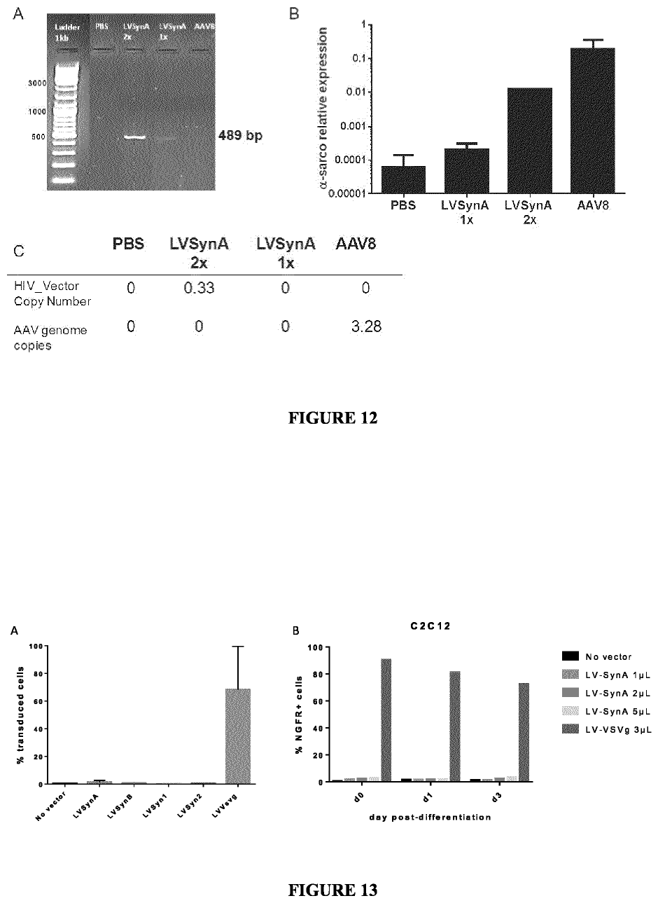

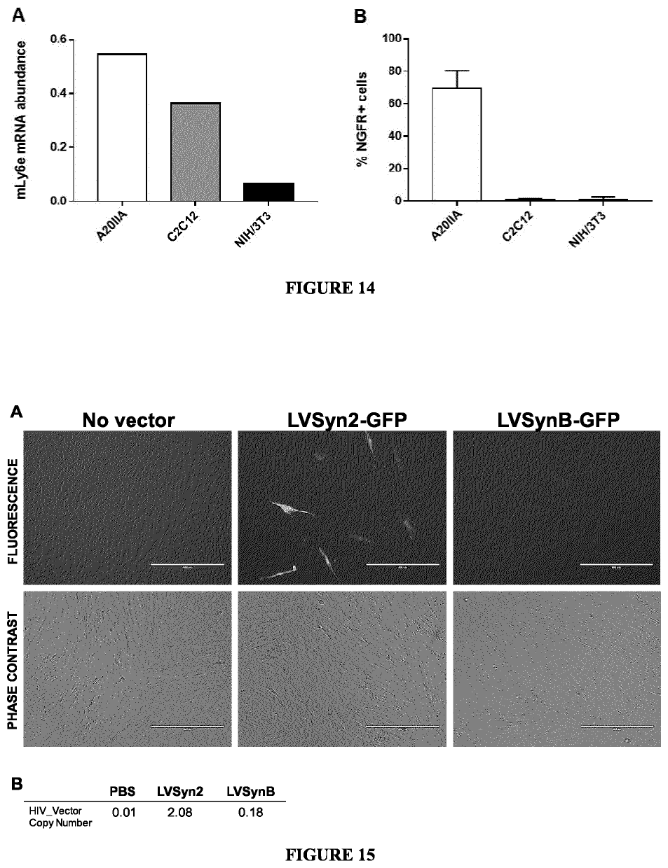

Representative photographs are shown and the bioluminescence of the right and left TA muscles is indicated below each photograph (FIG. 1). The results show that LV-SynA enable transgene expression locally in the muscle of MDX mice but not in C57Bl/6 controls and not in the liver of mice contrary to rAAV2/8. The signal in MDX mice injected intramuscularly with LV-SynA, represented here at 4 weeks post-injection shows that the gene transfer is stable and well-tolerated by the mouse. The signal was visible starting at day 6 post-injection. In contrast, no evidence of bioluminescence signal was observed at any time point examined in normal mice injected intramuscularly with LV-SynA. The signal obtained with the LV-SA-Luc2 is lower than with rAAV2/8-Luc2 but the rAAV2/8 vector, even though it was injected intramuscularly, disseminated much beyond muscle and was found at high levels in the liver, consistent with the known tropism of rAAV2/8 for mouse liver (Table I).

TABLE I
Comparison of bioluminescence obtained in the TA muscle or in liver of
normal or dystrophic mice following LV-SA Luc2 or rAAV2/8 injection.
Average Bioluminescence of TA and of liver (photons/sec) +/− SD (n)

| Signal | C57BL/6 | MDX |
|---|---|---|
| Muscle | | |
| TA-R (PBS injected) | 0.15 × 10⁵ ± | 0.04 × 10⁵ ± |
| | 0.25 (n = 8) | 0.02 (n = 7) |
| TA-L (LV-SA injected) | 0.69 × 10⁵ ± | 12.41 × 10⁵ ± |
| | 1.35 (n = 8) | 13.25 (n = 7) |
| Liver | | |
| LV-SA | 0.89 × 10⁵ ± | 0.39 × 10⁵ ± |
| | 0.12 (n = 8) | 0.07 (n = 7) |
| rAAV2/8 | 1174 × 10⁵ ± | 4030 × 10⁵ ± |
| | 193 (n = 4) | 1371 (n = 2) |

Expression of the bioluminescent transgene luciferase was quantified in liver and in the right and left Tibialis Anterior muscle. Average values, SD and number of mice tested (n) in 2 different protocols. Bioluminescence was measured 4 weeks after injection of vector. Quantification was done by drawing a mask to define the organ area based on the largest area detected by the highest signal. The same mask was applied to all mice from a same protocol. (*) statistically-significant difference between to LV-SynA-injected muscles of C57BL/6 mice and MDX mice (p = 0.03).

To ensure that the bioluminescent signal was due to luciferase and not to inflammation, and to confirm the presence of the transgene in muscle, immunohisto-chemistry was performed to localize the luciferase. FIG. 2 shows that luciferase was found inside muscle myofibers of mdx mice injected with the vector.

A summary of two experiments in which the bioluminescence signal of the TA was quantified shows that significantly higher levels of signal are found in mdx mice injected with the LV-SA vector compared to normal C57B16 mice (FIG. 3). Statistical analysis (Mann-Whitney) showed that the signal obtained with the LV-SA-Luc2 was significantly higher than in PBS (p=0.0009) and higher in mdx mice than in C57BL/6 mice (p=0.03).

Vector copy number in injected TA of MDX and normal mice was also measured by qPCR (FIGS. 4A and 4C) and the presence of integrated vector was verified by a more sensitive classical PCR (FIGS. 4B and 4D). The results confirm that the injection of syncytin-A-pseudotyped LV (LV-SynA) directly into muscle does not lead to a significant transduction of skeletal muscle tissue in normal mice (C57Bl/6; FIGS. 4A and 4B) but enables a significant transduction in MDX mice which constitute a model of Duchenne Muscular Dystrophy (FIGS. 4C and 4D).

To confirm that these findings could also apply to another dystrophic mouse model, luciferase gene transfer was tested in alpha-sarcoglycan-deficient mice (sgca−/− mice). Seven sgca−/− mice (6 week-old) were injected with the LV-SA luc2 vector in the left TA and with another vector in the right TA. An eighth mouse was used as negative controls. Results showed a clear bioluminescence signal in the muscle injected with the luciferase vector (FIG. 5 and Table II).

TABLE II
Bioluminescence obtained in the TA muscle of sgca −/−
dystrophic mice following LV-SA Luc2.

| | AverageBioluminescence |
|---|---|
| | (photons/sec) +/− SD (n) |
| TA-R (PBS injected) | 0.06 × 10⁵ ± |
| | 0.01 (n = 7) |
| TA-L (LV-SA injected) | 19.65 × 10⁵ ± |
| | 17.02 (n = 7) |

Expression of the bioluminescent transgene luciferase were quantified in Left and Right Tibialis Anterior. Average values, SD and number of mice tested (n) were obtained in 2 different protocols. Bioluminescence was measured 2 weeks after injection of vector. Quantification was done by drawing a mask to define the organ area based on the largest area detected by the highest signal. The same mask was applied to all mice from a same protocol. Mann-Whitney p value TAR vs TAL: p = 0.0006.

FIG. 6 shows that LV-SynA can be used to integrate detectable levels of a transgene cassette into skeletal muscle of alpha-sarcoglycan-deficient mice (sgca−/− mice). Comparably to MDX mice, the sgca−/− mice have a high regeneration rate of their skeletal muscle tissue. These data confirm the notion that LV-SynA vectors preferentially transduce regenerative muscle tissue. The data also show that LV-SynA vectors could be used to treat more than one dystrophic disease, possibly all dystrophic diseases in which high levels of skeletal muscle regeneration occur.

To determine if gene transfer could have a potential therapeutic interest, mdx mice were used to perform dystrophin exon skipping using a construction already reported by Goyenvalle et al. (Science, 2004, 306(5702):1796-9). The expression of the small nuclear RNA mex23 is an antisense sequence which will induce skipping of the exon 23 of the dystrophin gene which is mutated in the mdx mice and will permit the production of a slightly truncated dystrophin. A lentiviral vector pseudotyped with syncytin A and coding for the mex23 antisense sequence expressed from the U7 promoter (LV-SA U7mex23) was generated As control, we used the already described AAV1 vector coding for the U7-driven antisense mex23 sequence (Goyenvalle et al. Science 2004). The vectors were injected to mdx mice in the left TA. As controls, the right TA were injected with PBS or with a vector coding for Luc2. FIG. 7 shows that 2 weeks following injection of a LV-SA U7mex23 to mdx mice, it was possible to detect the presence of a 688 bp sequence corresponding to exon 23-skipped dystrophin RNA in the injected muscle and not in the control muscles. The LV-SA vector seems to be less efficient than the rAAV but additional experiments are needed to optimize vector dose and timing of detection.

### EXAMPLE 3: STABLE TRANSDUCTION AND REDUCED IMMUNOGENICITY ARE OBTAINED WITH LV-SYNA PARTICLES CONTRARY TO LV-VSVG

Materials and Methods

ELISPOT to Measure Transgene-Specific T Cell Immune Responses Induced by Gene Transfer

To measure transgene-specific immune responses, we used lentiviral vectors encoding the GFP-HY transgene described earlier (Ciré et al. Plos One 2014 PLoS One. 2014 Jul. 24; 9(7):e101644. doi: 10.1371/journal.pone.0101644. eCollection 2014). Cellular suspensions of erythrocyte-depleted spleen cells were obtained after the sacrifice of mice. IFN-γ enzyme-linked immunospot assays (ELISPOT) were performed by culturing 10⁶ spleen cells per well with or without 1 μM of Dby or Uty peptide in IFN-γ Enzyme-Linked Immunospot plates (MAHAS45, Millipore, Molsheim, France). As a positive control, cells were stimulated with Concanavalin A (Sigma, Lyon, France) (5 μg/ml). After 24 h of culture at +37° C., plates were washed and the secretion of IFNγ was revealed with a biotinylated anti-IFNγ anti-body (eBiosciences), Streptavidin-Alcalin Phosphatase (Roche Diagnostics, Mannheim, Germany), and BCIP/NBT (Mabtech, Les Ulis, France). Spots were counted using an AID reader (Cepheid Benelux, Louven, Belgium) and the AID ELISpot Reader v6.0 software. Spot forming units (SFU) are represented after subtraction of background values obtained with non-pulsed splenocytes.

Cytometric Bead Array (CBA) to Titrate Cytokines Induced by Transgene-Specific Immune Responses Following Gene Transfer

Stimulation media [medium, UTY (2 μg/mL), DBY (2 μg/mL), or Concanavalin A (5 μg/mL)] were plated and 10⁶ splenocytes/well were added. After 36 h of culture at +37° C., supernatants were frozen at −80° C. until the titration. Cytometric bead arrays were performed with BD Biosciences flex kits (IL-6, IFN-γ, TNFα, and RANTES). Briefly, capture bead populations with distinct fluorescence intensities and coated with cytokine-specific capture antibodies were mixed together. Next, 25 μL of the bead mix of beads was distributed and 25 μL of each sample (supernatants) was added. After 1 h of incubation at room temperature, cytokine-specific PE-antibodies were mixed together and 25 μL of this mix was added. After 1 h of incubation at room temperature, beads were washed with 1 mL of Wash buffer and data were acquired with an LSRII flow cytometer

Results

The stable transduction and reduced immunogenicity of LV-SynA particles were demonstrated in comparative assays with LV-VSVg.

The results show that LV-SynA provides long-lasting and stable gene transfer in MDX muscle whereas LV pseudotyped with other envelopes such as VSVg provide only temporary expression and the signal drops over time (FIG. 8). In addition, FIG. 8 confirms that LV-SynA cannot transduce normal skeletal muscle tissue at any time point. FIG. 8 suggests that perhaps long-term muscle progenitor cells, such as satellite cells, are transduced in MDX mice. Stable transgene expression was also observed following intramuscular delivery of a LV-SynA vector in sgca−/− mice and in MDX mice (FIG. 9). The data in MDX mice confirm those already shown in FIG. 8. LV-SynA vectors are less immunogenic than LV-VSVg vectors as they induced less transgene-specific immune responses when they are injected intramuscularly into MDX mice (FIG. 10). LV-VSVg vectors induce strong transgene-specific CD4 and CD8 T cell responses as measured by ELISPOT-IFNg (FIGS. 10A and 10B) and by the levels of infiltration of CD3+ T cells in the tissue (FIG. 10D). The reduced immune response obtained with LV-SynA vectors translated into higher levels of integrated vector in the tissue (FIGS. 10B and FIG. 10C).

The reduced immunogenicity of the LV-SynA vectors compared to LV-VSVg vectors was also observed following systemic administration (FIG. 11). Lower levels of transgene-specific CD4 and CD8 T cell responses (FIG. 11A) and lower levels of cytokines (FIG. 11B) are observed following intravenous injection of LV-SynA vector into normal mice compared to LV-VSVg.

### EXAMPLE 4: IN VIVO GENE DELIVERY OF A THERAPEUTIC GENE TO REGENERATING SKELETAL MUSCLE USING LV-SYNA PARTICLES

Materials and Methods

qPCR

qPCR AAV on AAV was determined according to the protocol described in example 2 using the following primers and probe:

AAV-Forward:
(SEQ ID NO: 22)
CCAGGCGAGGAGAAACCA
AAV-Reverse:
(SEQ ID NO: 23)
CTTGACTCCACTCAGTTCTCTTGCT
AAV-Probe:
(SEQ ID NO: 24)
CTCGCCGTAAAACATGGAAGGAACACTTC.

Quantification of Human Alpha-Sarcoglycan mRNA Following Gene Transfer in Mice

Total RNA was extracted from Tibialis Anterior (TA) muscle of tested mice following freezing and sectioning of the tissue, and extraction of RNA from frozen tissue sections with Trizol (Invitrogen). RNA was eluted in 25 μl RNase-free water and treated with Free DNA kit (Ambion) to remove residual DNA. Total RNA extracted for each sample was quantified by using a Nanodrop spectrophotometer (ND8000 Labtech, Wilmington Del.).

For quantification of the alpha-sarcoglycan expression, one μg of RNA was reverse-transcribed using the SuperScript II first strand synthesis kit (Invitrogen) and a mixture of random oligonucleotides and oligo-dT. Real-time PCR was performed using LightCycler480 (Roche) with 0.2 μM of each primer and 0.1 μM of the probe according to the protocol Absolute QPCR Rox Mix (ABgene). The primer pairs and Taqman probes used for the human α-sarcoglycan amplification were: 920hasarco. F: 5′-TGCTGGCCTATGTCATGTGC-3′ (SEQ ID NO: 25), 991hasarco.R:5′-TCTGGATGTCGGAGGTAGCC-3′ (SEQ ID NO: 26), and 946hasarco.P: 5′-CGGGAGGGAAGGCTGAAGAGAGACC-3′ (SEQ ID NO: 27). The ubiquitous acidic ribosomal phosphoprotein (P0) was used to normalize the data across samples. The primer pairs and Taqman probe used for P0 amplification were: m181P0.F (5′-CTCCAAGCAGATGCAGCAGA-3′; (SEQ ID NO: 28)), m267P0.R (5′-ACCATGATGCGCAAGGCCAT-3′; (SEQ ID NO: 29)) and m225P0.P (5′-CCGTGGTGCTGATGGGCAAGAA-3′; (SEQ ID NO: 30)). Results are expressed in fold change using the formula: relative abundance=2\hat{ }^{-ΔΔct} with ΔCt=Ct hasarco—Ct P0 and ΔΔCt=ΔCtsample−ΔCtcalibrator.

Results

The possibility to achieve gene transfer of a therapeutic gene using LV-syn2 vectors was demonstated with the human alpha sarcoglycan, in sgca−/− mice which are a model of limb girdle muscular dystrophy (FIG. 12)

The results shows also that repeated administration of the vector is possible to increase the levels of integrated vector (FIGS. 12A and 12C) and to increase transgene expression levels (FIG. 12B). The possibility to reinject to increase the dose is likely due to the low immune response obtained against the transgene following LV-SynA administration.

FIG. 12 shows that LV-SynA vectors achieve lower levels of copies and lower levels of transgene expression in muscle tissue compared to a rAAV vector. However the LV and rAAV have different characteristics and use different molecular mechanisms. LV-SynA vectors could be more advantageous in terms of persistency as they integrate stably into the genome of target cells contrary to rAAV which remain episomal. LV-SynA vectors could be used to treat people who cannot receive rAAV because they are seropositive for this vector. LV-SynA could also package transgenes of larger size as the cargo capacity of LV is about 10-13 Kb which is greater than 4.5 Kb for rAAVs.

### EXAMPLE 5: TRANSDUCTION OF REGENERATING MUSCLE CANNOT BE PREDICTED FROM IN VITRO DATA

Materials and Methods

Transduction of Murine and Human Myoblasts and Analyses by Flow Cytometry

C2C12 murine myoblats cell line was cultured in DMEM medium (Life Technologies) supplemented with 10% Foetal Bovine Serum or with 2% Horse Serum for the differenciation process. Transduction of cells was performed for 6 h with LV-Syn A or LV-VSVg vectors at 10⁵ or 5.10⁵ infectious genome per mL in presence of Vectofusin (12 μg/ml). Cellular mortality and transduction efficiency were evaluated, respectively, by 7-amino-actinomycin D labeling and measurement of NGFR or GFP expression using flow cytometry (FACS LSRII, BD Biosciences) after 3 or 5 days.

Ly6e mRNA Expression on Different Human and Murine Cell Lines and Murine Primary Cells.

mRNA from different murine cell lines (A20IIA, C2C12, NIH/3T3) and from total cells from the lung, spleen and bone marrow of C57BL/6 mice were extracted using the RNeasy® mini kit from Qiagen. The reverse transcription of the mRNA was performed using Verso cDNA synthesis kit from Thermofischer. A qPCR was performed on the cDNA using the following primers: mLy6e forward primer 5′ CGGGCTTTGGGAATGTCAAC 3′ (SEQ ID NO: 31), mLy6e reverse primer 5′ GTGGGATACTGGCACGAAGT 3′ (SEQ ID NO: 32), PO reverse primer 5′ CTCCAAGCAGATGCAGCAGA 3′ (SEQ ID NO: 33) and PO forward primer 5′ ACCATGATGCGCAAGGCCAT 3′ (SEQ ID NO: 34). PO was used as a warehouse gene. The abundance is calculated with the formula abundance=2-ΔCt.

Results

C2C12 cells which are murine myoblasts that are commonly used as a model of myoblast to myotube differentiation were transduced with LV-SynA and LV-VSVg vectors. When the cells cultured as replicative myoblasts were exposed to the vectors, only the LV-VSVg positive control achieved transduction (FIG. 13A). In FIG. 13B, the cells were exposed to the vector at different time points following the induction of differentiation into myotubes and to different doses of vector. Only the LV-VSVg positive control achieved transduction at every time point tested (FIG. 13B). FIG. 13 demonstrates that transduction of regenerating muscle cannot be predicted from in vitro data. FIG. 13 further confirms what is shown in FIG. 4, which is that not all types of muscle cells can be transduced with the LV-Syn vectors. The level of expression of mLy6e reported as the receptor for murine Syncytin A and the level of transduction with LV-Syncytin A vectors encoding ΔNGFR were compared on C2C12 cells and control cells (A20). The results show that the expression of mLy6e on muscle cells does not allow to predict the ability to transduce muscle cells by LV pseudotyped with SynA (FIG. 14). C2C12 cells express relatively abundant levels of Ly6e but are not transduced. FIG. 13 and FIG. 14 further confirms what is shown in FIG. 4, which is that not all types of muscle cells can be transduced with the LV-Syn vectors.

### EXAMPLE 6: IN VITRO TRANSDUCTION OF HUMAN SKELETAL MUSCLE MYOBLASTS CELLS WITH HUMAN SYNCYTIN 2 LV VECTORS

Materials and Methods

Transduction of Human Myoblasts and Analyses by Flow Cytometry

CSC-C3196 human skeletal muscle myoblasts cells (Creative Bioarray, Shirley, N.Y., USA) was cultured in collagen coated 24-wells plates and in Superculture Skeletal Muscle Cell culture medium supplemented with Fibroblast Growth Factor-2 (20 ng/mL). Transduction of cells was performed for 6 h with LV-Syn A or LV-VSVg vectors at 10⁵ or 5.10⁵ infectious genome per mL in presence of Vectofusin (12 μg/ml).

Cellular mortality and transduction efficiency were evaluated, respectively, by 7-amino-actinomycin D labeling and measurement of NGFR or GFP expression using flow cytometry (FACS LSRII, BD Biosciences) after 3 or 5 days.

Results

Human Syncytin 2 can be used to transduce human primary myoblasts (FIG. 14). Transgene expression (here GFP) is detected by microscopy in about 5-10% of the cells 5 days following infection with LV-Syn2 vectors (FIG. 14A). Analysis by qPCR confirmed the transduction and showed significant VCN obtained (FIG. 14B). LV-SYnB vectors provided some transduction but were much less efficient. These results show that LV pseudotyped with human syncytins such as Syncytin2 could be potentially used to transduce human skeletal muscle to express a transgene stably.

These results altogether show for the first time that syncytin—pseudotyped LV can be used to deliver genes preferentially in regenerative skeletal muscle. This is novel because LV are generally not used for gene transfer into muscle as they are either inefficient or immunogenic. These findings have therapeutic potential for Duchenne muscular dystrophy and potentially also for all myopathies involving a regenerative muscle phase such as for examples limb-girdle muscular dystrophies like alpha-sarcoglycan deficiency and others.

These results show also that LV-SA vectors behave very differently from rAAV, the gold-standard vector for gene delivery in muscle. While LV-syncytin vectors may generate lower vector copies and transgene levels than rAAV, there are 3 potential advantages for the LV-syncytin vectors to consider. First, the use of LV-SA in muscle remains local and does not appear to diffuse to other organs, limiting potential toxicity. This is not the case for rAAVs as seen in FIG. 1 with the rAAV8 going to the liver very effectively even if the administration was made into the muscle. Second, in vivo gene delivery with LV-Syncytin is expected to be more stable than with episomal rAAV due to the integrative nature of the LV vector and the lower immunogenicity of LV pseudotyped with syncytin. It is likely that LV vectors pseudotyped with syncytin which are more physiological than rAAV, due to the use of an envelope protein from an endogenous retrovirus, are less immunogenic than rAAV. Being less toxic to the liver, less immunogenic and more stable than rAAV, LV pseudotyped with syncytin can advantageously be administered repeatedly to achieve stable in vivo gene delivery without loss of transgene expressing cells. Third, LV have a larger cargo capacity than rAAV and can incorporate large transgenes such as dystrophin cDNA. In view of these advantages, LV pseudotyped with syncytin represents a very promising alternative to rAAV for gene therapy of myopathies.

1-17. (canceled)

18. A method of preventing and/or treating muscle injuries or diseases including regeneration phases as part of the disease physiopathological process in a patient in need thereof, comprising administering to the patient a therapeutically effective amount of a pharmaceutical composition targeting regenerating muscle tissue, comprising at least a therapeutic drug associated to a syncytin protein.

19. The method according to claim 18, wherein the syncytin protein is human or murine syncytin.

20. The method according to claim 19, wherein the syncytin is selected from the group consisting of human Syncytin-1, human Syncytin-2, murine syncytin-A and murine syncytin-B.

21. The method according to claim 18, wherein the drug and the syncytin protein are incorporated into particles.

22. The method according to claim 21, wherein the particles are selected from the group consisting of liposomes, exosomes, viral particles and virus-like particles.

23. The method according to claim 21, wherein the syncytin protein is displayed on the surface of the particles.

24. The method according to claim 21, wherein the particles are lentiviral or lentiviral-like particles pseudotyped with syncytin protein.

25. The method according to claim 18, wherein the drug is selected from the group consisting of therapeutic genes, genes encoding therapeutic proteins or peptides, therapeutic antibodies or antibody fragments, genome editing enzymes, interfering RNA, guide RNA for genome editing, antisense RNAs capable of exon skipping and drugs capable of stimulating muscle cell regeneration.

26. The method according to claim 21, wherein the drug is a gene of interest packaged into viral vector particles.

27. The method according to claim 18, wherein the drug is a gene of interest packaged into lentiviral vector particles pseudotyped with syncytin protein.

28. The method according to claim 27, wherein the syncytin is murine syncytin-A or human Syncytin-2.

29. The method according to claim 18, wherein the muscle injuries or diseases are selected from the group comprising: muscular dystrophy, dystrophinopathy, distal myopathy, myofibrillar myopathy, miscellaneous myopathy, myotonic syndrome, congenital myopathy, mitochondrial myopathy, metabolic myopathy, ion channel muscle disease, familial periodic paralysis, congenital myasthenic syndrome, neurogenic myopathy, auto-immune myopathy, lipid storage disease, hereditary cardiomyopathy, neuromuscular disorder, inflammatory myopathy, rhabdomyolysis, compartment syndrome or myoglobinuria, malignant hyperthermia, muscle infection, myofascial pain, muscle twitching, muscle injury produced by direct trauma, drug abuse, medication, toxic agents, ischemia, hot or cold temperature and physical exercise or overuse.

30. The method according to claim 18, wherein the muscle diseases are selected from the group comprising: muscular dystrophy, dystrophinopathy, distal myopathy, myofibrillar myopathy, miscellaneous myopathy, myotonic syndrome, congenital myopathy, mitochondrial myopathy, metabolic myopathy, ion channel muscle disease, familial periodic paralysis, congenital myasthenic syndrome, neurogenic myopathy, lipid storage disease, hereditary cardiomyopathy, neuromuscular disorders, myoglobinuria and malignant hyperthermia

31. The method according to claim 18, which is a method of gene therapy of the muscle diseases.

32. The method according to claim 18, wherein the drug is a gene of interest for therapy of muscle injuries or diseases selected from the group consisting of: DMD, MYOT, LMNA, CAV3, DES, DNAJB6, SGCA, SGCB, SGCG, SGCD, CAPN3, DYSF, TCAP, TRIM32, FKRP, POMT1, FKTN, POMGNT1, POMT2, ANDS, TTN, PLEC, EMD, FHL1, LMNA, SMN1, SMN2, PABPN1, NEB, ACTA1, TPM2, TPM3, TNNT1, CFL2, LMOD3, KBTBD13, KLHL40, KLHL41, RYR1, SEPN1, KBTKD13, MTM1, MTMR2, DUX4, FRG1, GYS1, GAA, GBE1, PYGM, PKFM, ALDOA, ENO3, GYG1 and functional variants thereof.

33. The method according to claim 18, wherein the pharmaceutical composition is for administration by injection.

34. A pharmaceutical composition targeting regenerating muscle tissue, comprising virus particles pseudotyped with syncytin protein, packaging a gene of interest selected from the group comprising: the genes DMD, MYOT, LMNA, CAV3, DES, DNAJB6, SGCA, SGCB, SGCG, SGCD, CAPN3, DYSF, TCAP, TRIM32, FKRP, POMT1, FKTN, POMGNT1, POMT2, ANO5, TTN, PLEC, EMD, FHL1, LMNA, SMN1, SMN2, PABPN1, NEB, ACTA1, TPM2, TPM3, TNNT1, CFL2, LMOD3, KBTBD13, KLHL40, KLHL41, RYR1, SEPN1, KBTKD13, MTM1, DUX4, FRG1, MTMR2, GYS1, GAA, AGL, PYGM, PKFM, ALDOA, ENO3, GYG1, functional variants thereof, interfering RNA, guide RNA for genome editing and antisense RNA capable of exon skipping, wherein the RNA target the gene of interest.

35. The pharmaceutical composition according to claim 34, wherein the virus particles are lentiviral vector particles.

36. A pharmaceutical composition targeting regenerating muscle tissue, comprising virus-like particles pseudotyped with syncytin protein, packaging a therapeutic RNA such as interfering RNA, guide RNA for genome editing and antisense RNA capable of exon skipping, said therapeutic RNA targeting a gene of interest selected from the group of genes comprising: DMD, MYOT, LMNA, CAV3, DES, DNAJB6, SGCA, SGCB, SGCG, SGCD, CAPN3, DYSF, TCAP, TRIM32, FKRP, POMT1, FKTN, POMGNT1, POMT2, ANO5, TTN, PLEC, EMD, FHL1, LMNA, SMN1, SMN2, PABPN1, NEB, ACTA1, TPM2, TPM3, TNNT1, CFL2, LMOD3, KBTBD13, KLHL40, KLHL41, RYR1, SEPN1, KBTKD13, MTM1, DUX4, FRG1, MTMR2, GYS1, GAA, AGL, PYGM, PKFM, ALDOA, ENO3 and GYG1.

37. The pharmaceutical composition according to claim 36, wherein the virus-like particles are lentivirus-like particles.

38. The pharmaceutical composition according to claim 36, wherein the syncytin protein is murine syncytin-A or human Syncytin-2.
